(19) United States
(12) Patent Application Publication
(10) Pub. No.: US 2025/0032425 A1
(43) Pub. Date: Jan. 30, 2025

(54) FIRST IN CLASS USE OF ESTROGEN RECEPTOR BETA AGONISTS IN TREATING ANGELMAN SYNDROME AND RELEVANT AUTISM SPECTRUM DISORDERS

(71) Applicant: BROWN UNIVERSITY, Providence, RI (US)

(72) Inventors: Yu-Wen Alvin Huang, Barrington, RI (US); Xin Yang, Providence, RI (US)

(21) Appl. No.: 18/781,151

(22) Filed: Jul. 23, 2024

Related U.S. Application Data
(60) Provisional application No. 63/515,204, filed on Jul. 24, 2023.

(51) Int. Cl. A61K 31/05; A61P 25/00

(52) U.S. Cl. CPC A61K 31/05; A61P 25/00

(57) ABSTRACT

The present disclosure provides, for instance, a method of treating oligodendroglial dysfunction including Angelman Syndrome and autism spectrum disorder, the method comprising administering to the subject a non-steroidal selective estrogen receptor beta agonist, thereby treating the oligodendroglial dysfunction in the subject.

### CROSS REFERENCE TO RELATED APPLICATIONS

This application claims the benefit of U.S. Provisional Application No. 63/515,204, filed Jul. 24, 2023. The contents of the aforementioned application are hereby incorporated by reference in their entirety.

### BACKGROUND

Angelman syndrome (AS) is a genetic disorder caused by loss of function of the Ube3A gene. Angelman syndrome and autism spectrum disorders share some characteristics, including developmental delay and speech issues. Dysfunction of oligodendrocytes, a type of supporting glial cell, may be one cause of cognitive symptoms in AS and autism spectrum disorder. There is a need in the art for new therapies that target oligodendroglial dysfunction.

### SUMMARY OF THE INVENTION

In some aspects, the present disclosure provides a method of treating Angelman syndrome in a subject, the method comprising administering to the subject an effective amount of a non-steroidal selective estrogen receptor beta agonist. In some aspects, the present disclosure provides a non-steroidal selective estrogen receptor beta agonist for use in the treatment of Angelman syndrome in a subject. In some aspects, the present disclosure provides the use of a non-steroidal selective estrogen receptor beta agonist for the manufacture of a medicament for the treatment of Angelman syndrome in a subject.

In some aspects, the present disclosure provides a method of treating autism spectrum disorder in a subject, the method comprising administering to the subject an effective amount of a non-steroidal selective estrogen receptor beta agonist. In some aspects, the present disclosure provides a non-steroidal selective estrogen receptor beta agonist for use in the treatment of autism spectrum disorder in a subject. In some aspects, the present disclosure provides the use of a non-steroidal selective estrogen receptor beta agonist for the manufacture of a medicament for the treatment of autism spectrum disorder in a subject.

In some embodiments, the non-steroidal selective estrogen receptor beta agonist has an EC50 for estrogen receptor beta of less than 100 nM, less than 50 nM, less than 20 nM, less than 15 nM, less than 10 nM, less than 9 nM, less than 8 nM, or less than 7 nM. In some embodiments, the EC50 of the non-steroidal selective estrogen receptor beta agonist for estrogen receptor alpha is 100 times greater, 200 times greater, 300 times greater, 400 times greater, 500 times greater, 750 times greater, or 1000 times greater than the EC50 of the non-steroidal selective estrogen receptor beta agonist for estrogen receptor beta.

In some embodiments, the non-steroidal selective estrogen receptor beta agonist comprises a compound of Formula I:

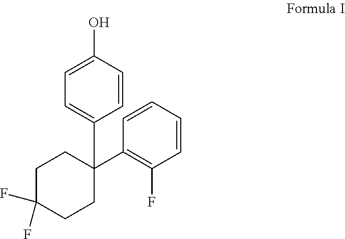 [SMILES: Oc1ccc(C2(c3ccccc3F)CCC(F)(F)CC2)cc1]

or a pharmaceutically acceptable salt thereof.

In some embodiments, the non-steroidal selective estrogen receptor beta agonist comprises a compound of Formula II:

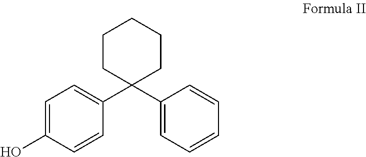 [SMILES: Oc1ccc(C2(c3ccccc3)CCCCC2)cc1]

or a pharmaceutically acceptable salt thereof.

In some embodiments, the non-steroidal selective estrogen receptor beta agonist comprises a compound of Formula III:

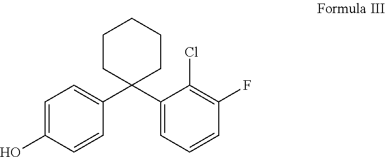 [SMILES: Oc1ccc(C2(c3cccc(F)c3Cl)CCCCC2)cc1]

or a pharmaceutically acceptable salt thereof.

In some embodiments, the non-steroidal selective estrogen receptor beta agonist comprises a compound of Formula IV:

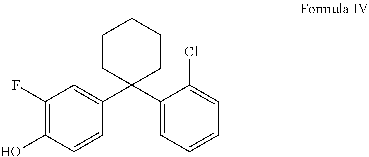 [SMILES: Oc1ccc(C2(c3ccccc3Cl)CCCCC2)cc1F]

or a pharmaceutically acceptable salt thereof.

In some embodiments, the non-steroidal selective estrogen receptor beta agonist is administered in a dose sufficient to reduce oligodendroglial dysfunction in the subject. In some embodiments, the non-steroidal selective estrogen receptor beta agonist is administered in a dose sufficient to delay onset of neurodevelopmental symptoms in the subject. In some embodiments, the non-steroidal selective estrogen receptor beta agonist is administered in a dose sufficient to reduce cognitive impairment symptoms in the subject. In some embodiments, the non-steroidal selective estrogen receptor beta agonist is administered in a dose sufficient to improve motor coordination in the subject. In some embodiments, the non-steroidal selective estrogen receptor beta agonist is administered in a dose sufficient to reduce motor learning defects in the subject.

In some embodiments, the non-steroidal selective estrogen receptor beta agonist is administered in a dose sufficient to maintain myelination levels or mitigate a decrease in myelination levels in the subject. In some embodiments, the non-steroidal selective estrogen receptor beta agonist is administered in a dose sufficient to promote proliferation of oligodendrocyte progenitor cells in the subject. In some embodiments, the non-steroidal selective estrogen receptor beta agonist is administered in a dose sufficient to promote differentiation of oligodendrocyte progenitor cells in the subject.

In some embodiments, the subject has a deletion in chromosome 15, e.g., in maternal chromosome 15. In some embodiments, the subject has a mutation in UBE3A. In some embodiments, the mutation is a truncation mutation.

In some embodiments, the subject has an age of 0-1 year, 1-2 years, 2-3 years, 3-4 years, 4-5 years, or greater than 5 years.

In some embodiments, the administration is oral administration, intraperitoneal injection, intravenous administration, or topical administration.

In some embodiments, the subject is identified as being at risk of developing Angelman syndrome. In some embodiments, the subject is identified as being at risk of developing autism spectrum disorder. In some embodiments, the subject is a human.

In some aspects, the present disclosure also provides a method of assaying whether an agent can increase KI67 expression in an OPC having reduced UBE3A expression. In some embodiments, the OPCs are iOPCs. In some embodiments, the OPCs are human OPCs. In some embodiments, the OPCs having reduced UBE3A expression were treated with an siRNA against UBE3A. In some embodiments, the agent is a small molecule. In some embodiments, the agent is a protein. In some embodiments, a plurality of agents (e.g., at least 10 or at least 20) are assayed. In some embodiments, KI67 levels are increased to the KI67 levels of a control cell, wherein the control cell is an otherwise similar cell that does not have reduced UBE3A expression (e.g., a cell not treated with an siRNA against UBE3A).

### BRIEF DESCRIPTION OF THE DRAWINGS

FIGS. 1A-1B depict reduced OPC (oligodendrocyte progenitor cell) population and myelination in Angelman Syndrome (AS) mouse brains compared to wild type (WT). FIG. 1A (left): fluorescence microscopy images of brain sections from 1-month-old AS and wild type (WT) littermate control mice were stained with Olig2 (red), Ube3A (green), and nucleus (DAPI, blue). FIG. 1A (right): bar plots depicting quantification of oligodendroglia (Olig2+ cells) and their expression of Ube3A (green) in oligodendroglia (Olig2+ cells) from WT and AS mouse brains showing a reduction in oligogendroglial cell density in AS animals compared to WT. FIG. 1B (left): images of brain sections from 1 month old and 6 month old WT and AS mice stained with myelination marker MBP (black). FIG. 1B (right): bar plots depicting measurements of gray matter (cortex thickness, top) and white matter (corpus callosum thickness, middle) structures and myelination (MBP+ area, bottom), showing a reduction in myelination in AS animals compared to WT. For FIGS. 1A-1B, n=3-6 animals, statistical analysis done by Two-way ANOVA post hoc by Dunnett's multiple comparisons test, *p<0.5. ** p<0.05. *** p<0.001.

FIGS. 2A-2D depict the results of a drug screen for the rescue of iOPCs (induced oligodendrocyte progenitor cells) proliferation impaired by Ube3A depletion. FIG. 2A: fluorescence microscopy images showing impaired OPC proliferation by Ube3A depletion assayed with EdU incorporation (green), immune staining of KI67 (blue), Ube3A (red), and nucleus (DAPI, blue) in control (siNC, left) and Ube3A knockdown (siUbe3A, right) iOPCs. FIG. 2B: immunoblotting of KI67, Ube3A, Olig2, and B3-tublin for siNC- and siUbe3A-treated iOPCs. FIG. 2C: a plot depicting quantification of KI67 immunoblotting signals of Ube3A knockdown iOPCs treated with approximately 20 therapeutic compounds (10 uM final dose) normalized to response of control siRNA (siNC=1.0). Statistically significant compounds: BDNF, FGF and AC-186. FIG. 2D: a plot showing EC50 analysis of estrogen receptor β agonists AC-186 and DPN. For FIGS. 2A-2D, n=4-11 experiments; statistical analyses done by One-way ANOVA post hoc by Dunnett's multiple comparisons test, *P<0.05, ** P<0.01, *** P<0.001, **** P<0.0001.

FIG. 2E depicts AC-186 ameliorating proliferation impairments by Ube3a removal.

FIGS. 3A-3B depict the rescue of impaired proliferation in primary rat OPCs deficient of Ube3A by AC-186. FIG. 3A: microscopy images of primary rat OPCs purified from the cortexes of 7 day old pups, (left) brightfield image and (right) stained with OPC marker PDGFRa (green), Ube3A (red), and DAPI (blue). FIG. 3B: immunoblotting (left) and a plot showing quantification (right) of KI67 and Ube3A protein levels in siNC- and siUbe3A-treated primary rat OPCs in cultures, showing rescue of OPC proliferation in Ube3A knockdown OPCs with AC-186. For FIGS. 3A-3B, n=3-6 experiments; statistical analysis done by One-way ANOVA post by Dunnett's multiple comparisons test, *P<0.05, ** P<0.01, *** P<0.001, **** P<0.0001.

FIGS. 4A-4C depict the expression of estrogen receptors altered by Ube3A depletion in iOPC. FIG. 4A: fluorescence microscopy images showing the expression of all 3 major estrogen receptors in iOPCs, estrogen receptor alpha (ESRα), beta (ESRβ) and GPER. FIG. 4B: immunoblotting (left) and a plot showing quantification (right) of UBE3A, GPER, ESRα, ESRβ, and Ki67 protein in control and Ube3A knockdown iOPCs, showing that Ube3A knockdown downregulates ESRB (the specific receptor for AC-186) and upregulates GPER in iOPCs. FIG. 4C: gel and quantification of KI67 protein level showing that knockdown of ESRβ, but not ESRα or GPER, reduces OPC proliferation marked by KI67 protein level, mimicking Ube3A knockdown.

FIGS. 5A, 5B, and 6 depict the role of Ube3a in OPC lineage.

FIGS. 7A-7C and 8 depict Ube3A depletion impairing iOPC proliferation.

FIG. 9 depicts Ube3a depletion in primary rat OPCs impairing proliferation.

FIG. 10 depicts reduced expression of Ube3A and Ki67 in iOPCs from AS patient derived iPSCs.

FIGS. 11A and 11B depict reduced population and myelination in AS mouse brains.

FIG. 12A depicts mRNA expression of Ube3a and estrogen receptors in iOPCs with or without Ube3a siRNA (SiUbe3a).

FIG. 12B depicts expression of ESRs in various induced cells with Ube3a depleted.

FIG. 13 depicts expression of estrogen receptors, showing that ESRB depletion mimics Ube3a removal.

FIG. 14 depicts ESR expression in AS brains.

FIG. 15 depicts evidence consistent with co-expression of Ube3a and ESR in OPCs.

FIG. 16 depicts the clasping score and results of rotarod training in WT and AS mice treated with saline (control) or AC-186. Top row of stars depict significance of AS-AC186 compared to AS-Saline. Middle row of stars depict significance of WT-Saline compared to AS-Saline. Bottom row of stars depict significance of WT-AC186 compared to AS-Saline.

FIG. 17 depicts the results of fear conditioning in WT and AS mice treated with saline or AC-186.

FIGS. 18A-18G depict compound screening to uncover the mechanism behind impaired OPC proliferation due to UBE3A depletion. FIG. 18A: Immunofluorescent staining shows UBE3A expression in iPSC-derived oligodendrocyte precursor cells (iOPCs) marked by NG2. Olig2 and DAPI, but devoid of negative control GFAP. FIG. 18B: Left-Immunofluorescent staining to assay the OPC proliferation altered by UBE3A knockdown, measuring the expression of two independent proliferation markers, Ki67 (magenta) expression and EdU (green) nuclear incorporation, in iOPCs treated with non-targeting control siRNA (siNC) or siRNA against UBE3A (siUBE3A) and labeled with DAPI (blue) and UBE3A (red); Right-data were plotted by cell numbers positive for either or both markers (normalized to control, siNC=1.0). iOPCs treated with siUBE3A showed a decrease in both EdU and Ki67 staining. n=40-48 regions of interest from 3 independent experiments. FIG. 18C: Immunoblotting for measurement of Ki67 expression and PDGFRa activation (Y754 phosphorylation) in iOPCs subject to RNAi-mediated UBE3A knockdown by siNC or siUBE3A. Both UBE3A and Ki67 were decreased in iOPCs treated with siUBE3A compared to the negative control, while PDGFRa and p-PDGFRa were measured at similar levels. n=7 experiments. FIG. 18D: Compound screening identified agents capable of restoring OPC proliferation reduced by UBE3A loss. Forty-five compounds (listed in Table 1) were individually tested at 10u M over 24 hours on UBE3A-deficient iOPCs with around a 50% reduction in Ki67 expression. The potential of these compounds to reinstate Ki67 expression, relative to vehicle control (DMSO), was quantified via immunoblotting, with Ki67 levels normalized to beta-tubulin and compared to the siNC group (value=1.0). AC-186, an estrogen receptor-β (ERB) agonist: BDNF, brain-derived neurotrophic factor; FGF, fibroblast growth factor were identified as capable of restoring Ki67 expression. n=3-10 per condition. FIG. 18E: Immunofluorescence to confirm the effect of AC-186 on OPC proliferation, with EdU incorporation in iOPC nuclei measured across three conditions: siNC plus vehicle, siUBE3A plus vehicle, and siUBE3A plus AC-186 at 10 μM for 24 hours. Cell counts positive for EdU were normalized to the siNC group (siNC=1.0), with n=24-28 region of interest from 3 independent experiments. AC-186 increased EdU staining in cells treated with siUBE3A. FIG. 18F: Assessment of multiple ESRB agonists with distinct chemical structures, including AC-186, WAY-200070, DPN, LY500307, Liquiritigenin and E2 (Table 1), for their effects on OPC proliferation. Each compound's selectivity was noted, especially the preference of AC-186 for ESRB over ESRα, with n=3-9 per condition. FIG. 18G: Left-Immunofluorescent staining to assay the OPC proliferation altered by UBE3A depletion followed by ESRβ stimulation, measuring the EdU (red) nuclear incorporation in iOPCs expressing Cas9 and a sgRNA targeting UBE3A via lentiviral transduction (Lenti-sgUBE3A #1-3) or a control sgRNA (Lenti-sgCtrl) and treated with either the vehicle control (DMSO), AC-186, DPN, or LY500307 commenced at a standard concentration of 10 μM, and then labeled with DAPI (blue) and UBE3A (green); Right-data were plotted by the percentage of cells (DAPI) positive for EdU. n=4 experiments. AC-186, DPN, and LY500307 increased the number of EdU+ cells after treatment with any of the sgRNAs. As applicable, scale bars represent 100 μm. Statistical analyses were conducted using Student's t-test for FIG. 18C. One-way ANOVA for FIGS. 18B, 18D, 18E and 18F, and Two-way ANOVA for FIG. 18G. Post-hoc tests were performed where applicable, with significance levels indicated as follows: * p<0.05. ** p<0.005. *** p<0.001. **** p<0.0001. ‘ns’ denotes not significant. Selected comparisons are highlighted as necessary.

FIGS. 19A-19I depict rescue of OPC proliferation impairment due to UBE3A depletion through ESR-β activation. FIG. 19A: Schematic illustrating the process of generating iPSC-derived oligodendrocyte lineage cells using a refined protocol from Vulakh and Yang, 2024 (Vulakh, G., and Yang, X. (2023). Characterizing the Neuron-Glial Interactions by the Co-cultures of Human iPSC-Derived Oligodendroglia and Neurons. Methods Mol. Biol. 2683. 10.1007/978-1-0716-3287-1_9). The human iPSCs employed in this study were the extensively characterized KOLF2.J1 control reference line, sourced from the INDI (Ramos, D. M., Skarnes, W. C., Singleton, A. B., Cookson, M. R., and Ward, M. E. (2021). Tackling neurodegenerative diseases with genomic engineering: A new stem cell initiative from the NIH. Neuron 109, 1080-1083. 10.1016/j.neuron.2021.03.022). Initially, iPSCs are converted into neural precursor cells (iNPCs, marked by Nestin) through SMAD inhibition in the first week. These iNPCs are then induced into oligodendrocyte precursor cells (iOPCs, marked by PDGFRa and A2B5) by the second week. Subsequently, iOPCs differentiate into pre-myelinating oligodendrocytes (pre-iOLs, marked by O4) by the third week and finally mature into oligodendrocytes (OLs) by weeks 4-5, when myelination assays are performed. FIG. 19B: qPCR for UBE3A mRNA expression along oligodendroglial differentiation, at the stages of iNPC, iOPC, pre-iOL and iOL; plotted relative to iOPC(=1.0) after normalizing UBE3A mRNA transcript levels to GAPDH levels. n=3 experiments. FIG. 19C: Data from single-cell RNA-sequencing of mouse oligodendrocyte lineage cells, as reported by Marques et al. 2016 (Oligodendrocyte heterogeneity in the mouse juvenile and adult central nervous system. Science (New York, N.Y.) 352, 1326-1329. 10.1126/science.aaf6463), showcasing the expression patterns of UBE3A across different stages of oligodendrocyte differentiation in both juvenile and adult central nervous systems. FIG. 19D: Immunoblotting showing no change in cell death of iOPCs undergoing RNA-mediated UBE3A knockdown, using the apoptosis-inducer staurosporine (5 μM for 2h) as a positive control: protein levels of UBE3A and Cl-Caspase were first normalized to β-actin (ACTB) and plotted as relative to the control condition (siNC=1.0). n=6 experiments. FIG. 19E: Immunoblotting analysis demonstrating the relationship between UBE3A protein levels and cell proliferation in iOPCs; left panel, by adjusting the dosage of siRNA targeting UBE3A (siUBE3A). Middle Panel: meta-analysis of immunoblotting data for UBE3A and the proliferation marker Ki67 in iOPCs treated with varying doses of siUBE3A. Intensity values were normalized to a loading control and compared against the normalized control (siNC), set as 1.0. A significant positive correlation between UBE3A and Ki67 levels is depicted. Right panel: data presented as a bar graph, categorizing groups based on the degree of UBE3A reduction achieved by RNA-mediated knockdown. Groups exhibiting less than 80% of the mean UBE3A level relative to siNC demonstrated notably lower Ki67 expression compared to the group with the smallest reduction (80-100% of siNC mean). Sample sizes were n=30 for <20% reduction, 20 for 20-40% reduction, 21 for 40-60% reduction, 11 for 60-80% reduction, and 28 for 80-100% reduction, across multiple experiments. FIG. 19F: Cross-platform validation of the efficacy of AC-186 using rat primary OPCs deficient in UBE3A treated with siNC or siUBE3A and either vehicle or AC-186, 10 μM over 24 hours, across four conditions. Densitometric analysis of UBE3A and Ki67. normalized to β-tubulin (TUBB1), was compared to siNC plus vehicle (value=1.0), with n=4 per condition. Rat OPCs treated with siUBE3A and AC-186 showed increased Ki67 expression relative to cells treated with siUBE3A alone. FIG. 19G: Efficacy of ESRβ agonists in mitigating proliferation deficits in UBE3A-depleted OPCs was verified by immunoblotting (left), assessing the levels of 2 distinct proliferation markers, Ki67 and PCNA, post-treatment with AC-186, DPN, and LY500307 (10 μM, 24 hours). Right protein levels were normalized to GAPDH and compared to siNC plus vehicle (set at 1.0). AC-186, DPN, and LY500307 increased expression of Ki67 and PCNA in cells treated with siUBE3A. n=4-9 per group. FIG. 19H: Baseline effects of AC-186 on OPC proliferation analyzed by Ki67 expression in untreated iOPCs exposed to vehicle or drugs (AC-186 or DPN; 10 μM, 24 hours), with densitometric signals of immunoblotting normalized to GAPDH. No significant changes were observed by One-way ANOVA, complemented by post-hoc tests. n=7 experiments. FIG. 19I: Establishment of the half-maximal effective concentration (EC50) for AC-186 and DPN, which reflects the potency of these compounds in reversing the proliferation deficits of UBE3A-deficient OPCs, by applying a range of drug concentrations to iOPCs treated with siUBE3A for 24 hours. Ki67 expression was measured as a marker of proliferation using immunoblotting. The EC50 calculations, which indicate the concentration at which each drug achieves a 50% maximal response, were derived from the dose-response curves. These curves plot Ki67 expression levels, normalized to the siNC plus vehicle group (set as 1.0), against drug concentrations. The EC50 values were determined to be approximately 16 nM for AC-186 and 19 nM for DPN, revealing their relative effectiveness in a cellular context mimicking UBE3A loss. As applicable: Scale bars were set at 100 μm. Statistical analyses were executed with One-way ANOVA with post-hoc analyses, with significance levels denoted by *p<0.05. ** p<0.005, *** p<0.001, **** p<0.0001. Non-significant results are labeled as ‘ns’. Selected comparisons are highlighted as necessary.

FIGS. 20A-20E depict the identification of ESR-β signaling pathway in regulating OPC proliferation impaired by UBE3A depletion. FIG. 20A: Immunofluorescence to track estrogen receptor (ESR) expression in OPCs, by staining of all 3 ESRs, ESR-α, ESR-β and GPER (top panels), in iOPCs co-labeled with UBE3A (middle panels) and DAPI; scale bar, 100 μm. ESR-α, ESR-β and GPER all showed expression in iOPCs. FIG. 20B: Immunoblotting to assess OPC expression of individual ESRs, by analyzing protein levels of ESR-α, ESR-β and GPER in iOPCs treated with siNC or siUBE3A, normalized to GAPDH and expressed relative to the siNC condition (set as 1.0). After treatment with siUBE3A, expression of ESR-β was decreased, while expression of GPER was increased. n=5-6 experiments. FIG. 20C: The activation of the ERK/MAPK signaling pathway in OPCs, particularly downstream of ESR-β, was analyzed in iOPCs subjected to 6 knockdown conditions: siNC, siUBE3A, siESRα, siESRβ, and combinations of siUBE3A with siESRα or siESRβ. These were followed by treatment with vehicle control or the ESR-β agonist AC-186 (10 μM, 30 min). The activation of ERK and its substrate S6 was determined by the ratio of phosphorylated to total ERK and S6 levels, adjusted relative to the vehicle control group (standard value of 1.0). ERK phosphorylation and S6 phosphorylation was decreased in cells treated with siESRβ, or siESRβ and siUBE3A combined. n=3 experiments. FIG. 20D: Immunoblotting to evaluate the role of ERK/MAPK activation in the restoration of OPC proliferation following ESR-β activation, with the addition of ERK phosphorylation inhibitors U0126 or PD98059 to UBE3A-depleted OPCs, with or without AC-186 co-treatment (10 μM. 24 hours). The quantification of Ki67 expression and UBE3A levels, being normalized to β-tubulin (TUBB1) and compared to the siNC condition (value of 1.0). n=5 experiments. FIG. 20E: A schematic depicting the regulatory role of ESR-β signaling pathway in OPC proliferation, identified by combining genetic knockdown techniques and pharmacological interventions. Activation of ESR-β selectively triggers the ERK/MAPK pathway, as shown by ERK phosphorylation and the ERK substrate S6 activation, essential for cell proliferation. Reduction in ESR-β function due to UBE3A loss can be compensated by ESR-β agonists, suggesting a mechanism to improve impaired OPC proliferation. As applicable: statistical significance was determined using One-way ANOVA followed by post-hoc comparisons, with selected comparisons presented. Significance levels marked as *p<0.05. ** p<0.005. *** p<0.001. **** p<0.0001, ns not significant, across relevant comparisons.

FIGS. 21A-21C depict the role of UBE3A depletion on estrogen receptor (ESR) signaling and downstream pathway activation in neural cells. FIGS. 21A and 21B: Immunoblotting assessed how UBE3A depletion impacts ESR expression in neurons, astrocytes and microglia. Protein levels of ESR-α, ESR-β, GPER, and UBE3A were quantified in human iPSC-derived neurons (IN), astrocytes (iAs) and microglia (iMG) after siNC or siUBE3A treatment, normalized against GAPDH, and compared to siNC (value set at 1.0). GPER expression was increased in iAs after siUBE3A treatment. n=4-5 replicate experiments; statistical significance was determined by Two-way ANOVA with subsequent post-hoc tests; ** p<0.01. **** p<0.0001, ns not significant. FIG. 21C: Determination of how UBE3A depletion affects downstream signaling of ESRs. Top-Immunoblotting was used to measure the activation of several key signaling pathways in iOPCs treated with siNC or siUBE3A. Bottom-Pathway activation was assessed by calculating the ratio of phosphorylated to total protein levels for mediators such as 4EBP1, ERK, S6, TrkB, and NFkB. The efficiency of UBE3A knockdown was verified, and changes in activation status were normalized to GAPDH and compared to siNC (baseline set at 1.0). Levels of pERK and pS6 were decreases in cells treated with siUBE3A. Four replicates were analyzed for each condition, with statistical significance assessed using Two-way ANOVA and subsequent post-hoc tests; ** p<0.01. **** p<0.0001.

FIGS. 22A-22G depict oligodendroglial homeostasis and learning behaviors after ESR-β activation in a UBE3A-Deficient AS mouse model. FIG. 22A: Brain sections from wildtype (WT) and Angelman syndrome (AS) mice at early postnatal days (P7-10) were subjected to immunofluorescence to examine oligodendrocyte populations and UBE3A expression in the hippocampus CA1 region. Olig2 (green) and myelin basic protein (MBP, red) were stained to identify oligodendrocyte lineage cells, co-stained with UBE3A (magenta), and nuclei visualized with DAPI (blue). Scale bar: 100 μm. FIG. 22B: Quantification of oligodendrocyte populations and ESRβ expression. Cell densities for Olig2-positive and O4-positive oligodendrocytes were calculated (left and middle), and ESRβ expression was quantified by fluorescence intensity within Olig2-positive cells. In AS brain sections, the number of Olig2-positive and O4-positive cells/mm² was reduced, and the expression of ESRβ in Olig2-positive cells was decreased compared to wild type. Data from 3 animals per group were analyzed. Scale bars: 100 μm in left panel, 25 μm in right panel. FIG. 22C: Timeline for ESRβ agonist AC-186 efficacy study in AS mice. Juvenile WT or AS mice received daily intraperitoneal injections of ESRβ agonist AC-186 (10 mg per kg of body weight) or a vehicle solution starting at P13-14. Behavioral assessments commenced three weeks later over six days: Hindlimb Clasping (P35; d0), Rotarod (d1-3), and Fear Conditioning (d4-6) tests, commenced. Post-behavioral analysis (P42), mice were sacrificed for immunofluorescence studies of brain sections to quantify oligodendroglial cell density, estrogen receptor expression, and myelination status. Investigative groups were WT with vehicle, WT with AC-186, AS with vehicle, and AS with AC-186. FIG. 22D: Scoring of Hindlimb Clasping. Black symbols, male; blue symbols, females. Hindlimb clasping decreased in AS animals treated with AC-186 compared to AS animals treated with the vehicle. The number of animals per group was 20 for WT-vehicle, 11 for WT-AC186, 13 for AS-vehicle, and 11 for AS-AC186. FIG. 22E: The motor coordination and learning assessed by the Rotarod test, measuring the time until mice fell or were dislodged from the rotating barrel across all four groups, WT with vehicle (black), WT with AC-186 (blue), AS with vehicle (red) and AS with AC-186 (green), from days 1-3. Time to fall increased in AS animals treated with AC-186 compared to AS animals treated with the vehicle. FIG. 22F: The results of Contextual Fear Conditioning, presented as the percentage of time spent freezing post-tone/shock. In contextual fear conditions, the percentage of time spent freezing post-tone/shock increased in AS animals treated with AC-186 compared to AS animals treated with the vehicle. FIG. 22G: Examination of OPC population and proliferation in wildtype and AS mouse brains after treatment with vehicle or AC-186; Left-brain sections were immunostained for OPC marker PDGFRa (top), proliferation marker Ki67 (middle) and nuclear marker DAPI (bottom) to visualize total and proliferating populations of OPCs in the corpus callosum; OPC population quantified as their density, calculated by the number of PDGFRa-positive and DAPI-stained OPCs per square millimeter of corpus callosum area (left bar graph); Right-OPC proliferation is measured as the proportion of dividing OPCs (indicated by Ki67 and PDGFRa dual positivity) relative to the total OPC count (right bar graph). OPC density and proliferating OPCs increased in AS animals treated with AC-186 compared to AS animals treated with the vehicle. n=6-10 animals per group. As applicable: Statistical significance assessed using Student's t-test (FIG. 22B), One-way ANOVA (FIGS. 22D, 22F and 22G), or Two-way ANOVA (FIG. 22E) and subsequent post-hoc tests; * p<0.05. ** p<0.01. *** p<0.001, ** p<0.0001, ns not significant.

FIGS. 23A-23D depict diminished UBE3A levels in oligodendrocytes across developmental stages in AS mice. FIG. 23A: Examination of oligodendrocyte populations and UBE3A expression in the corpus callosum and fimbria of fornix in brain sections from WT and AS pups (P7-10). Left panels, immunofluorescence staining was performed using Olig2 and myelin basic protein (MBP) markers, with concurrent UBE3A labeling and DAPI for nuclear visualization. Scale bars 300 μm. Right panels, A magnified view of the immunofluorescence-stained sections highlighted in the designated areas (white boxes), detailing the white matter regions from both wildtype and AS pups. Scale bar. 100 μm. FIG. 23B: Density metrics of oligodendrocytes within the corpus callosum and fimbria were derived by counting cells positive for Olig2 (left, corpus callosum) or MBP (middle, corpus callosum; right, fimbria of fornix). Cell densities were plotted against the assessed area for each marker. Olig2-positive cells were reduced in the corpus callosum in AS brain sections compared to WT. MBP positive cells were reduced in the corpus callosum and the fimbria in AS brain sections compared to WT. FIG. 23C: Assessment of oligodendroglial populations and UBE3A protein levels in hippocampal CA1 sections of WT and AS juveniles at postnatal day 30 (P30). Left panels: oligodendrocytes were identified with Olig2 (red), UBE3A was labeled in green, and nuclei were counterstained with DAPI (blue). Scale bars indicate 100 μm. Right panels: enlarged imaging of the white boxed in the leftmost panels highlight Olig2-positive cells and their UBE3A expression, providing a detailed comparison of oligodendrocytes in wildtype and AS juvenile brain sections. Scale bar, 50 μm. FIG. 23D: The oligodendroglial cell density and UBE3A expression were quantified across developmental milestones: pups (P7-10), juveniles (P30), and adults (P180). The data reflect the number of Olig2-positive oligodendrocytes per area, with UBE3A intensity normalized against wildtype levels (set at 1.0). Both Olig2-positive cells and UBE3A intensity in Olig2-positive cells were decreased across developmental stages in AS animals compared to WE animals. n=3-8 animals per group. As applicable: Statistical analyses were performed using Student's t-test (FIG. 23B) or Two-Way ANOVA (FIG. 23D) complemented with post-hoc tests, with significance denoted by *p<0.05. ** p<0.005. *** p<0.001 and **** p<0.0001 when comparing AS to WT groups.

FIGS. 24A-24D depict improved oligodendroglial homeostasis and myelination in treatment-responsive AS mice. FIG. 24A: Immunofluorescence for analysis of oligodendroglia population and ESRβ expression in WT and AS mice treated with vehicle control or AC-186, by imaging the corpus callosum stained with Olig2, O4, ESRβ and DAPI. Scale bar: 30 μm. FIG. 24B: Calculations of Olig2-positive and O4-positive oligodendroglial cell densities and their ESRβ expression in the corpus callosum among four groups: WT with vehicle, WT with AC-186, AS with vehicle, and AS with AC-186. Olig2-positive and O4-positive cells/mm² increased in AS mice treated with AC-186 compared to AS mice treated with saline. ESRβ intensity in Olig2-positive and O4-positive cells also increased in AS mice treated with AC-186 compared to AS mice treated with saline. n=5-14 animals per condition. FIG. 24C: Comparative assessment of oligodendroglial populations and myelination across brain regions of motor cortex (left column), hippocampus (right column), shown in FIG. 24C page 1, and fimbria of fornix (left column), shown in FIG. 24C page 2, by quantifying the cell densities of Olig2-positive (top row) and O4-positive (middle row) cells as well as the MBP immunofluorescence signal intensity (bottom row). Olig2-positive, O4-positive, and MBP intensity increased in AS mice treated with AC-186 compared to AS mice treated with saline in the cortex, hippocampus, and fimbria. n=5-14 per condition. FIG. 24D: Evaluation of UBE3A expression in oligodendrocyte lineage cells and neuron-like cells, based on morphology and lack of oligodendroglial markers Olig2 and MBP. UBE3A intensity was comparable in AS mice treated with AC-186 compared to AS mice treated with saline. The bars, from left to right, represent WT-vehicle; WT-AC186; AS-Vehicle; and AS-AC186. n=5-14 per condition. As applicable: One-way ANOVA with post-hoc tests was used to determine statistical significance; * p<0.05. ** p<0.005. *** p<0.001. **** p<0.0001, ns not significant.

FIGS. 25A-25I depict the role of UBE3A via ESR-β signaling on OPC proliferation and oligodendrocyte differentiation. FIG. 25A: qPCR to evaluate the differentiation and maturation for myelination of iOPCs after persistent UBE3A knockdown, by measuring mRNA transcripts of UBE3A and myelination markers MBP and PLP1 in iOPCs treated with siNC or siUBE3A and differentiated in vitro. MBP and PLP1 mRNA expression decreased in iOLs treated with siUBE3A. n=3 independent experiments. FIG. 25B: In vitro myelination assays analyzed the effect of persistent UBE3A knockdown on iOPCs differentiation and maturation in nano-fiber-based cultures, with immunofluorescent staining of MBP (magenta) and DAPI (blue) and imaging of nano-fibers (grey). Quantification by the area (left graph) and density (right graph) of MBP fluorescent signals (normalized to siNC=1.0). MBP area and intensity decreased in cells treated with siUBE3A. n=3 experiments. Scale bar: 25 μm. FIG. 25C: Immunoblotting was conducted to assess the impact of UBE3A depletion on ESR-β expression during oligodendrocyte differentiation. Protein levels of ESR-β in UBE3A-deficient iOPCs and subsequently differentiated iOLs were quantified, normalized against the loading control GAPDH, and expressed relative to the iOPC+siNC control condition (set at 1.0). ESRβ expression decreased in iOPCs treated with siUBE3A compared to iOPCs and iOLs treated with siNC, or iOLs treated with siUBE3A. n=4 experiments. FIG. 25D: The effects of AC-186 (10 M) and clemastine (2.5 μM) on OPC proliferation (24 hours) and oligodendrocyte differentiation (3 days) were assessed via immunoblotting. Levels of Ki67 and Myelin Associated Glycoprotein (MAG) were measured in UBE3A-deficient iOPCs and iOLs differentiated from them, normalized against GAPDH, and compared to the iOPC+siNC condition. n=4 experiments. FIG. 25E: Immunoblotting to examine the role of UBE3A for OPC self-renewal and oligodendrocyte differentiation across different developmental stages. Expression of maturation and myelination markers (MBP, MAG, PLP1, CNPase) was quantified following UBE3A depletion timed from iOPCs to pre-iOLs, normalized against TUBB1. and compared to the iOPC+siNC control condition (set at 1.0). n=4 experiments. FIG. 25F: Imaging validation of iOPCs derived from an AS patient with a UBE3A truncation mutation (AS iOPCs), compared to multiple human pluripotent stem cell lines. OPC markers NG2 and PDGFRa and proliferation markers Ki67 and EdU were immunofluorescently labeled, and OPC proliferation was quantified by the ratio of Ki67+ or EdU+ cells to total OPC cells, in comparison to control iOPCs generated from human embryonic stem cells H1 and H9 and the reference iPSC line Kolf2 lines. n=12 regions of interest from 3 independent experiments. The ratio of Ki67 positive cells and EdU positive cells was lower in iOPCs derived from the AS patient compared to control cell lines. FIG. 25G: Immunofluorescence analysis to characterize the response of AS iOPCs to AC-186 treatment (10 μM, 24 hours) on proliferation, quantified by the ratio of proliferating (EdU+) cells. The ratio of EdU positive iOPCs derived from the AS patient increased when treated with AC-186 compared to treatment with vehicle. n=3 experiments. FIG. 25H: Immunoblotting to assess the effect of restoring UBE3A expression on the proliferation of UBE3A-deficient OPCs. In AS iOPCs, UBE3A expression was re-introduced via lentiviral transduction (Lv-UBE3A, 3 days), and Ki67 levels were quantified, normalized to β-tubulin (TUBB1), and compared to a control condition using an empty lentiviral vector (Lv-Ctrl). Ki67 levels increased in AS iOPCs transduced with Lv-UBE3A. n=10 experiments. FIG. 25I: The effects of restoring UBE3A expression and treatments with AC-186 or clemastine on oligodendrocyte differentiation in a UBE3A loss-of-function context were evaluated. AS iOPCs were differentiated into iOLs and treated with lentiviral transduction of UBE3A, AC-186 (10 μM, 72 hours) or clemastine (2.5 μM, 72 hours), with the expression levels of maturation and myelination markers (MAG, CNPase, MBP) quantified. n=3 experiments. Applied tests include Student's t-test (FIGS. 25B, 25G, and 25H), One-way ANOVA (FIGS. 25C, 25D, 25E, 25F, and 25I), and Two-way ANOVA (FIG. 25A) with post-hoc analyses and selected comparisons indicated. Significance levels marked as *p<0.05. ** p<0.005. *** p<0.001. **** p<0.0001, ns not significant, across relevant comparisons.

FIGS. 26A-26F depict evaluation of UBE3A and ESR signaling in oligodendrocyte differentiation for myelination. FIG. 26A-26B: Immunoblotting assessed oligodendrocyte differentiation in iOPCs, following the protocol outlined in FIG. 19A. Protein expression levels of myelination markers myelin basic protein (MBP), myelin associated glycoprotein (MAG), 2′,3′-cyclic nucleotide 3′ phosphodiesterase (CNPase), and proteolipid protein 1 (PLP1) were measured. Results were normalized to GAPDH and are presented as fold change relative to the untreated iOPC condition, set as 1.0. MBP expression increased as cells differentiated. MAG, CNPase, and PLP1 expression also increased as cells differentiated. n=4 experiments. FIG. 26C: Immunoblotting to investigate the effects of AC-186 (10 UM) and clemastine (2.5 M) on oligodendrocyte differentiation, with timing adjustments for UBE3A depletion. These treatments were applied as cells transitioned from iOPCs to pre-iOLs and subsequently to myelin-producing iOL stages. Expression levels of MAG and CNPase were normalized to TUBB1 (β-tubulin) and plotted as fold change relative to the siNC control condition (set as 1.0). n=4 experiments. FIG. 26D: Immunoblotting to evaluate the differentiation potential of UBE3A-deficient OPCs (AS iOPCs), generated from AS patient with UBE3A loss-of-function truncation (c.1540delC) (pVal515serfs*3A), into myelinating cells by quantifying the expression of UBE3A and 4 oligodendrocyte markers (MBP, MAG, PLP1, CNPase). Data were normalized against GAPDH and expressed relative to control iOLs derived from the reference iPSC line Kolf2. The AS cells had decreased expression of UBE3A, MAG, CNP, MBP, and PLP1 relative to the Kolf2 cells. n=4 experiments. FIG. 26E: Immunoblotting to measure estrogen receptor subtypes ESR-α, ESR-β, and GPER in AS-derived iOPCs, normalizing against TUBB1 and comparing to iOPCs derived from Kolf2, as well as H1 and H9 human embryonic stem cell lines, used as references. Results are presented as fold changes relative to the Kolf2-derived iOPCs (set as 1.0). AS cells had lower expression of ESRβ and higher expression of GPER compared to control cells. n=3 experiments. FIG. 26F: Immunoblotting to analyze the proliferation effect of AC-186 (10 μM, 24 hours) on AS-derived OPCs, assessed by quantifying Ki67 expression levels, normalized against β-tubulin (TUBB1), and expressed as fold change relative to the vehicle control condition. AS cells treated with AC-186 showed increased Ki67 expression compared to cells treated with vehicle. n=3 experiments. As applicable: Statistical significance was determined using Student's t-test (FIG. 26F) and One-way ANOVA (FIGS. 26A, 26C, 26D and 26E) followed by post-hoc comparisons, with selected comparisons presented. Significance levels were indicated with asterisks *p<0.05, ** p<0.005, *** p<0.001, **** p<0.0001, with “ns” denoting non-significance.

### DETAILED DESCRIPTION OF THE INVENTION

In the following description, for an explanation, numerous specific details provide a thorough understanding of the compositions and methods disclosed herein. However, it may be evident that the compositions and methods may be practiced without these specific details. Aspects, modes, embodiments, variations, and features of the compositions and methods are described below in various levels of detail to provide a substantial understanding of the present disclosure.

### Definitions

For convenience, the meaning of some terms and phrases used in the specification, examples, and appended claims, are listed below. Unless otherwise defined, all technical and scientific terms have the same meaning as commonly understood by a person having ordinary skill in the biomedical art to which this invention belongs. A term's meaning provided in this specification shall prevail if any apparent discrepancy arises between the meaning of a definition provided in this specification and the term's use in the biomedical art.

The singular forms a, an, and the like include plural referents unless the context dictates otherwise. For example, a reference to a cell comprises a combination of two or more cells.

As used herein, the term “comprising” means that other elements can also be present in addition to the defined elements presented. Using comprising indicates inclusion rather than limitation.

As used herein, the term “consisting essentially of” means the listed elements are required for a given embodiment. The term permits additional elements that do not materially affect the basic and functional characteristics of that embodiment of the invention.

As used herein, the term “consisting of” means compositions, methods, and respective components thereof, exclusive of any element not recited in that description of the embodiment.

As used herein, the term “effective amount” refers to the amount sufficient to cause beneficial or desired results, for example, clinical results, and, as such, an “effective amount” depends upon the context in which it is being applied. Many ways are known in the biomedical art to determine the effective amount for an application. For example, pharmacological methods for dosage determination can be used in the therapeutic context. In therapeutic or prophylactic applications, the amount of a composition administered to the subject depends on the type and severity of the disease and the characteristics of the individual, such as general health, age, sex, body weight, tolerance to drugs, and on the degree, severity, and type of disease. Persons having ordinary skill in the biomedical art can determine appropriate dosages depending on these and other factors. In some embodiments, an effective amount results in inhibition of a target protein by at least 5%, 10%, 20%, 30%, 40%, 50%, 60%, 70%, 80%, 90%, or more. In some embodiments, an effective amount results in a reduction of the size of a tumor by at least 5%, 10%, 20%, 30%, 40%, 50%, 60%, 70%, 80%, 90%, or more.

As used herein, the term “estrogen receptor beta agonist” refers to an agent that specifically binds an estrogen receptor beta protein and activates downstream signaling. In some embodiments, the agent comprises a molecule or a complex. In some embodiments, the agent is exogenous. In some embodiments, the agent is a full agonist. In some embodiments, the agent is a co-agonist. In some embodiments, the agent is a partial agonist. In some embodiments, the agonist is a small molecule.

As used herein, the term “expression” refers to the transcription or translation of a particular nucleic acid sequence driven by a promoter. In some embodiments, expression refers to the level of accumulation of an RNA. In some embodiments, expression refers to the accumulation of a protein.

As used herein, the term “nucleic acid” refers to a polymeric molecule incorporating units of ribonucleic acid, deoxyribonucleic acid, or an analog thereof. In some embodiments, the nucleic acid is in single stranded form. In some embodiments, the nucleic acid is in double stranded form. In some embodiments, the nucleic acid is genomic DNA, cDNA, or RNA (e.g. mRNA). In some embodiments, the nucleic acid contains analogues of natural nucleotides that have similar binding properties as the reference nucleic acid. In some embodiments, the nucleic acid containing analogues of natural nucleotides are metabolized in a manner similar to naturally occurring nucleotides.

As used herein, the term “or” refers to and/or. The term and/or as used in a phrase such as A and/or B herein includes both A and B; A or B; A (alone); and B (alone). Likewise, the term and/or as used in a phrase such as A, B, and/or C encompasses each embodiment: A, B, and C; A, B, or C; A or C; A or B; B or C; A and C; A and B; Band C; A (alone); B (alone); and C (alone).

As used herein, the terms “peptide.” “polypeptide,” and “protein” are used interchangeably, and refer to a molecule comprised of two or more amino acid residues covalently linked by peptide bonds between the alpha-amino and carboxy groups of adjacent residues. In some embodiments, the polypeptide comprises a modified amino acid. In some embodiments, the polypeptide refers to a natural peptide, a recombinant peptide, or a combination thereof. In some embodiments, the polypeptide refers to short chains of amino acids. In some embodiments, the polypeptide refers to long chains of amino acids. In some embodiments, the polypeptide refers to a biologically active fragment, a substantially homologous polypeptide, an oligopeptide, a variant of a polypeptide, a modified polypeptide, a derivative, an analog, or a fusion protein. A person having ordinary skill in the biomedical art recognizes that individual substitutions, deletions, or additions to a peptide, polypeptide, or protein sequence which alters a single amino acid or a small percentage of amino acids in the encoded sequence are a conservatively modified variant where the alteration results in the substitution of amino acid with chemically similar amino acid and retains the desired activity of the polypeptide. Such conservatively modified variants also do not exclude polymorphic variants, interspecies homologs, and alleles consistent with the disclosure.

As used herein, the term “selective”, when referring to an agent that binds estrogen receptor beta, refers to an agent that exhibits an affinity for estrogen receptor beta that is 100 times stronger than that which it exhibits for estrogen receptor alpha.

As used herein, the term “subject” refers to a mammal, including but not limited to a dog, cat, horse, cow, pig, sheep, goat, chicken, rodent, or primate. Subjects can be house pets (e.g., dogs, cats), agricultural stock animals (e.g., cows, horses, pigs, chickens, etc.), laboratory animals (e.g., mice, rats, rabbits, etc.), but are not so limited. Subjects include human subjects. The human subject may be a pediatric, adult, or geriatric subject. The human subject may be of either sex. In some embodiments, the subject may have a condition or disease or be at risk of developing a condition or disease.

As used herein, the terms “treat”, “treatment” and “treating” refer to the reversal, alleviation, amelioration, inhibition, reduction, slowing or halting of the progression, severity and/or duration of a disease, disorder, or medical condition, or the amelioration of one or more symptoms of a disease, disorder, or medical condition. In some embodiments, the disease, disorder, or medical condition is a proliferative disorder, such as growth of a tumor. In some embodiments, the terms “treat.” “treatment,” and “treating” refer to the amelioration of at least one measurable physical parameter of the disease, disorder, or medical condition not necessarily discernible by the patient. In some embodiments, the terms “treat”, “treatment” and “treating” refer to the inhibition of the progression of disease, disorder, or medical condition, either physically by, e.g., stabilization of a discernible symptom, physiologically by, e.g., stabilization of a physical parameter, or both. In some embodiments, the terms “treat”, “treatment” and “treating” refer to the reduction or stabilization of tumor size or cancerous cell count. Treatment is effective, for example, if one or more symptoms or clinical markers are reduced or if the progression of a condition is reduced or halted. Treatment includes not just the improvement of symptoms or markers but also a cessation or at least slowing of progress or worsening of symptoms expected absent treatment.

This invention is not limited to the particular methodology, protocols, reagents, etc., described herein and as such can vary.

The disclosure described herein does not concern a process for cloning humans, processes for modifying the germ line genetic identity of humans, uses of human embryos for industrial or commercial purposes, or processes for modifying the genetic identity of animals likely to cause them suffering with no substantial medical benefit to man or animal, and animals resulting from such processes.

### Non-Steroidal Estrogen Receptor Beta Agonists

In some aspects, the present disclosure provides a method of treating Angelman syndrome in a subject, the method comprising administering to the subject an effective amount of a non-steroidal selective estrogen receptor beta agonist.

In some aspects, the present disclosure provides a method of treating autism spectrum disorder in a subject, the method comprising administering to the subject an effective amount of a non-steroidal selective estrogen receptor beta agonist.

### AC-186

In some embodiments, the non-steroidal selective estrogen receptor beta agonist comprises AC-186 or a pharmaceutically acceptable salt thereof. AC-186 has a chemical structure according to Formula I:

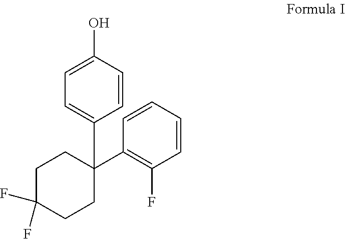 [SMILES: Oc1ccc(C2(c3ccccc3F)CCC(F)(F)CC2)cc1]

### AC-131

In some embodiments, the non-steroidal selective estrogen receptor beta agonist comprises AC-131 or a pharmaceutically acceptable salt thereof. AC-131 has a chemical structure according to Formula II:

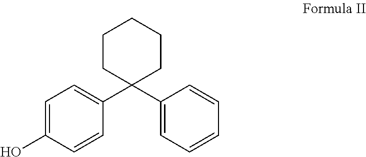 [SMILES: Oc1ccc(C2(c3ccccc3)CCCCC2)cc1]

### AC-623

In some embodiments, the non-steroidal selective estrogen receptor beta agonist comprises AC-623 or a pharmaceutically acceptable salt thereof. AC-623 has a chemical structure according to Formula III:

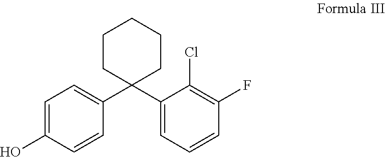 [SMILES: Oc1ccc(C2(c3cccc(F)c3Cl)CCCCC2)cc1]

### AC-957

In some embodiments, the non-steroidal selective estrogen receptor beta agonist comprises AC-957 or a pharmaceutically acceptable salt thereof. AC-957 has a chemical structure according to Formula IV:

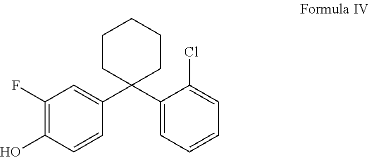 [SMILES: Oc1ccc(C2(c3ccccc3Cl)CCCCC2)cc1F]

### Diarylpropionitrile

In some embodiments, the non-steroidal estrogen receptor beta agonist comprises diarylproprionitrile (DPN) or a pharmaceutically acceptable salt thereof. DPN has a chemical structure according to Formula V:

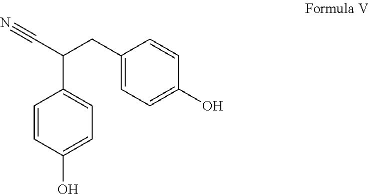 [SMILES: N#CC(Cc1ccc(O)cc1)c1ccc(O)cc1]

### WAY-200070

In some embodiments, the non-steroidal estrogen receptor beta agonist comprises WAY-200070 or a pharmaceutically acceptable salt thereof.

### LY500307 (Erteberel)

In some embodiments, the non-steroidal estrogen receptor beta agonist comprises LY500307 (Erteberel) or a pharmaceutically acceptable salt thereof. LY500307 has a chemical structure according to Formula VI:

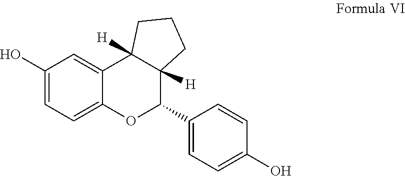 [SMILES: Oc1ccc([C@@H]2Oc3ccc(O)cc3[C@@H]3CCC[C@@H]32)cc1]

### Estrogen Hormone

In some aspects, the present disclosure provides a method of treating Angelman syndrome in a subject, the method comprising administering to the subject an effective amount of an estrogen hormone.

In some aspects, the present disclosure provides a method of treating autism spectrum disorder in a subject, the method comprising administering to the subject an effective amount of an estrogen hormone.

In some embodiments, the estrogen hormone comprises estradiol (E2) or a pharmaceutically acceptable salt thereof.

### GPR30 Antagonists

In some aspects, the present disclosure provides a method of treating Angelman syndrome in a subject, the method comprising administering to the subject an effective amount of a GPR30 antagonist.

In some aspects, the present disclosure provides a method of treating autism spectrum disorder in a subject, the method comprising administering to the subject an effective amount of a GPR30 antagonist.

In some embodiments, the GPR30 antagonist comprises G-15 or a pharmaceutically acceptable salt thereof.

### Growth Factors

In some aspects, the present disclosure provides a method of treating Angelman syndrome in a subject, the method comprising administering to the subject an effective amount of a growth factor.

In some aspects, the present disclosure provides a method of treating autism spectrum disorder in a subject, the method comprising administering to the subject an effective amount of a growth factor.

In some embodiments, the neurotrophic factor is fibroblast growth factor (FGF).

### Neurotrophic Factors

In some aspects, the present disclosure provides a method of treating Angelman syndrome in a subject, the method comprising administering to the subject an effective amount of a neurotrophic factor.

In some aspects, the present disclosure provides a method of treating autism spectrum disorder in a subject, the method comprising administering to the subject an effective amount of a neurotrophic factor.

In some embodiments, the neurotrophic factor is brain-derived neurotrophic factor (BDNF).

### Other Agents

In some embodiments, the present disclosure provides a method of treating Angelman syndrome in a subject, the method comprising administering to the subject an effective amount of retinoic acid, rolipram, clemastine, ketoconazole, PDGFα-AA, azathioprine, SUN-11602, fingolimod, FRAX, ezatiostat, siponimod, Liquiritigenin, or D-Syn3.

In some embodiments, the present disclosure provides a method of treating autism spectrum disorder in a subject, the method comprising administering to the subject an effective amount of retinoic acid, rolipram, clemastine, ketoconazole, PDGFα-AA, azathioprine, SUN-11602, fingolimod, FRAX, ezatiostat, siponimod, Liquiritigenin, or D-Syn3.

### Methods of Treatment

In some embodiments, the methods described herein are used to treat Angelman syndrome. Angelman syndrome is a genetic disorder. In some embodiments, Angelman syndrome is caused by a mutation in the UBE3A gene, e.g., a deletion, insertion, or point mutation. In some embodiments, Angelman syndrome is caused by the loss of function of the maternal copy of the UBE3A gene. In some embodiments, Angelman syndrome is caused by a deletion in chromosome 15, e.g., in maternal chromosome 15. In some embodiments, Angelman syndrome is an oligodendroglial disorder. In some embodiments, Angelman syndrome is associated with abnormal myelination. In some embodiments, the brain is affected. In some embodiments, Angelman syndrome causes developmental delays. In some embodiments, developmental delays are apparent after 6 months of age, e.g., from 6-12 months of age. In some embodiments, Angelman syndrome causes seizures, e.g., epilepsy. In some embodiments, Angelman syndrome causes severe language impairment with little or no speech. In some embodiments, Angelman syndrome causes movement and balance problems (e.g., ataxia). In some embodiments, Angelman syndrome causes a small head size (e.g., microcephaly). In some embodiments, Angelman syndrome causes sociable behavior with frequent smiling.

In some embodiments, the methods described herein are used to treat autism spectrum disorder. In some embodiments, autism spectrum disorder includes a disorder chosen from autism, fragile X syndrome, Rett syndrome (RTT), autism disorders, Asperger's syndrome, childhood disintegrative disorders and unspecified Pervasive Developmental Disorder Not Otherwise Specified (PDD-NOS) and Pathological Demand Avoidance (PDA). In some embodiments, autism spectrum disorder co-occurs with Angelman syndrome.

### EXAMPLES

### Example 1: UBE3A Governs Proliferation and Differentiation of Oligodendrocyte Precursor Cells In Vitro

This example demonstrates that knockdown of UBE3A in iPSC-derived OPCs (iOPCs) impairs proliferation and maturation of iOPCs. iOPCs may be generated using methods found in, e.g., Assetta, B. et al. “Generation of human neurons and oligodendrocytes from pluripotent stem cells for modeling neuron-oligodendrocyte interactions” J Vis Exp 2020.

The iOPC generation demonstrated a full yet accelerated development course for assaying a wide spectrum of oligodendroglial functions (see, e.g., Assetta, B. et al. “Generation of human neurons and oligodendrocytes from pluripotent stem cells for modeling neuron-oligodendrocyte interactions” J Vis Exp 2020). The knockdown of UBE3A leads to impaired proliferation and delayed maturation of control iOPCs (data not shown.)

### Example 2: Angelman Syndrome Model Mice Show a Reduced Oligodendrocyte Precursor Population

This example demonstrates that AS mice show a reduced oligodendrocyte precursor population in the brain compared to WT mice. AS mouse models are described, e.g., in Rotaru, D et al., “Angelman Syndrome: From mouse models to therapy” Neuroscience 2020 and Yang, X. “Towards an understanding of Angelman syndrome in mice studies” J Neurosci Res 2020.

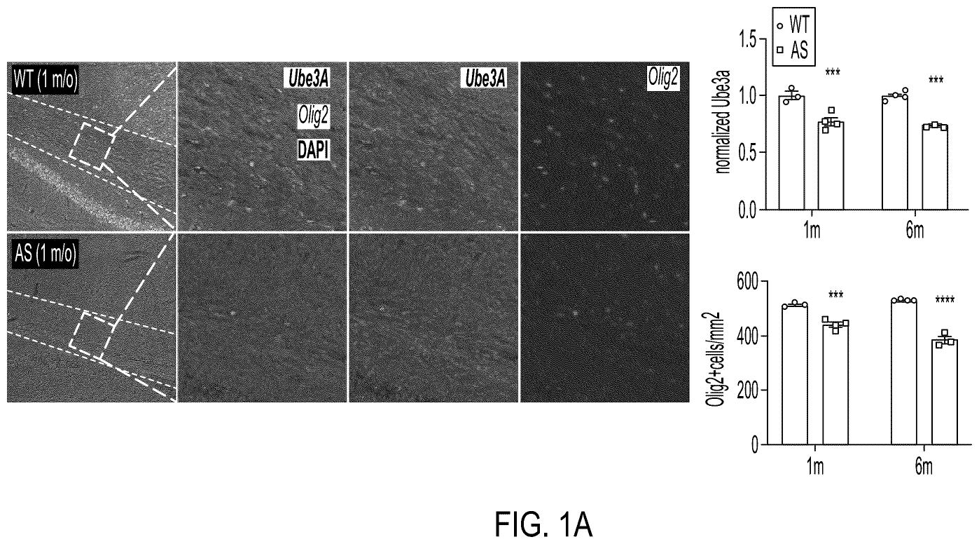

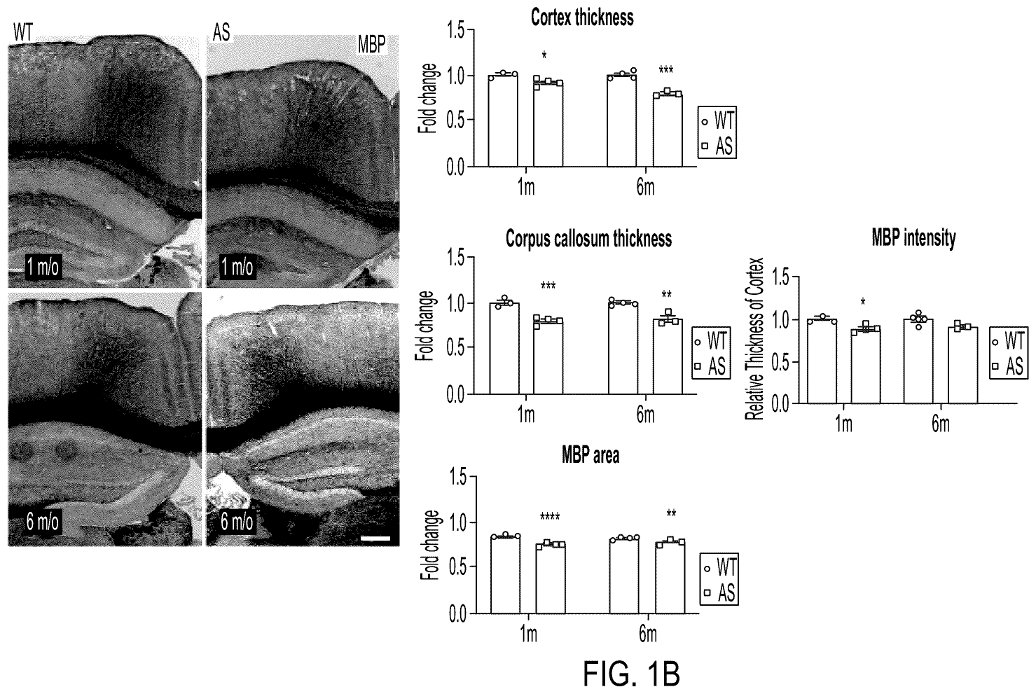

FIG. 1A shows a reduced number of Olig2+ cells in AS mouse brain sections stained for Ube3A, Olig2+, and DAPI compared to WT mouse brain sections. When quantified, the amount of Ube3A protein staining was significantly lower in AS mouse brain sections compared to WT mouse brain sections at both 1 and 6 months of age. Similarly, the number of Olig2+ cells/mm² was significantly lower in AS mouse brain sections compared to WT mouse brain sections at both 1 and 6 months of age.

### Example 3: Angelman Syndrome Model Mice Show Reduced Myelination

This example demonstrates that AS mice show reduced myelination compared to WT mice at 1 and 6 months of age.

Myelination is a crucial developmental process of the brain that is performed by oligodendrocyte lineage cells and is critical for proper cognitive function. In Angelman Syndrome (AS), myelination has been well documented to be deficient throughout infancy to young adulthood, the critical time window for cognitive development.

FIG. 1B shows a reduction in MBP staining in AS mouse brain sections compared to WT mouse brain sections. When quantified, AS mouse brains showed a statistically significant reduction in cortex thickness, corpus callosum thickness, and MBP+ area at both 1 and 6 months of age.

### Example 4: Characterization of Development and Differentiation of Angelman Syndrome Patient Derived iOPCs

iOPCs derived from Angelman syndrome patient-derived iPSC lines will be prepared. These iPSCs carry UBE3A loss-of-function mutations. The effect of AS mutations on the timing of oligodendroglial differentiation will be characterized by contrasting control and AS human oligodendrocyte lineage cells with the morphology and expression of gene markers representing stages of iOPC (D3-4, pre-myelinating (D14) and mature oligodendrocyte (D21). In addition, an in vitro myelination assay (D21-28) will be performed to study the oligodendroglial functionality in AS patient derived iOPCs.

### Example 5: A Drug Screen for the Rescue of iOPC Proliferation Impaired by Ube3A Depletion

This example describes a drug screen wherein selected neuroprotective reagents were tested in human iOPCs. The drug screen revealed a class of estrogen receptor (ER) agonists, led by AC-18613-15 (a highly selective ER-β agonist), is able to rescue the defective iOPC proliferation and myelinating differentiation caused by Ube3A loss of function.

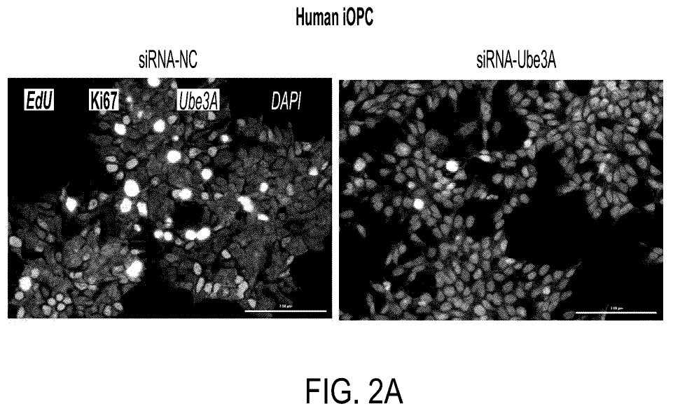

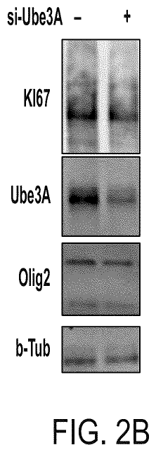

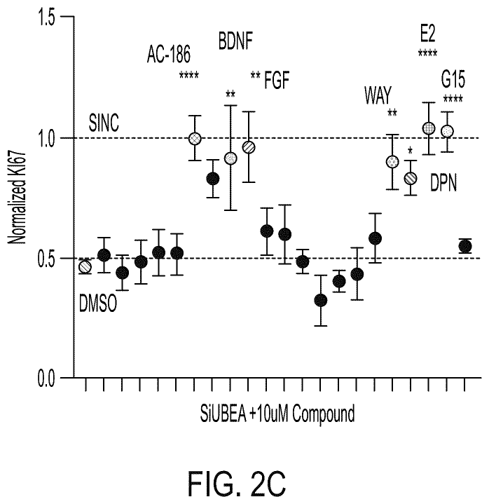

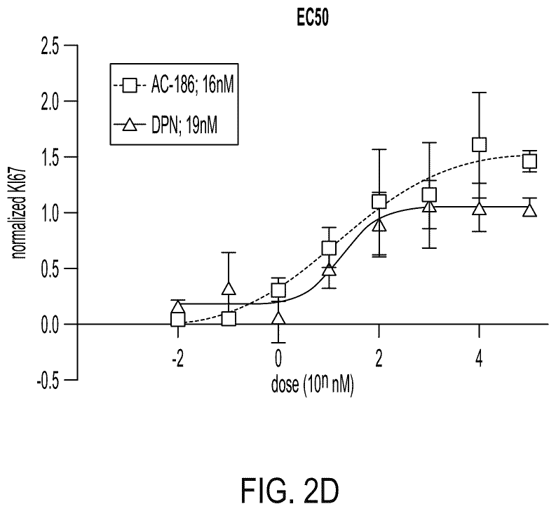

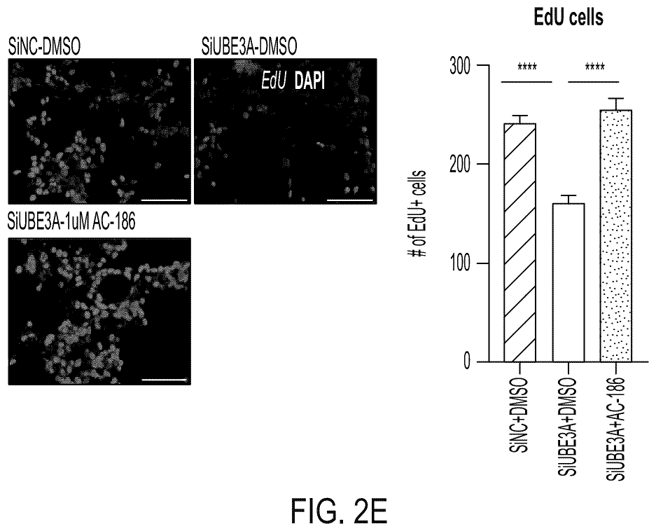

Out of the first round of drug screening based on 22 curated neuroprotective small molecules, it was repeatedly observed that brain-derived neurotrophic factor (BDNF), fibroblast growth factor (FGF) and AC-186 restored the KI67 expression in UBE3A siRNA-treated iOPCs to the level of control iOPCs (FIG. 2C-2D). Further analyses to compare AC-186 and a relevant compound Diarylpropionitrile (DPN, an ERB agonist of previous generation that once reached Phase 2 clinical trial), showed that the half maximal effective concentration (EC50) for the rescue of the effect of Ube3A loss on Ki67 expression was around 16 nM for AC-186 and 19 nM for DPN (FIG. 2D).

### Example 6: Treating iOPCs Derived from Subjects with Angelman Syndrome with Drug Screen Hits

iOPCs derived from Angelman syndrome patient-derived iPSC lines will be prepared. These iPSCs carry UBE3A loss-of-function mutations. The iOPCs will then be treated with a suitable dosage of AC-186 and DPN, e.g., as described in Example 5. Responses in estrogen receptor signaling and oligodendroglial functions, including iOPC proliferation and differentiation into mature oligodendrocytes, will be examined and compared to control iOPCs derived from iPSC lines from healthy individuals, as described in, e.g., Example 5. An increase in AS patient derived iOPC proliferation and differentiation will indicate that AC-186 and/or DPN can rescue iOPC proliferation and differentiation in the genetic context of human cells derived from Angelman syndrome patients.

### Example 7: Rescue of Impaired Proliferation in Primary Rat OPCs Deficient of Ube3A by AC-186

This example demonstrates that AC-186 rescues impaired proliferation in primary OPCs with Ube3A siRNA knockdown.

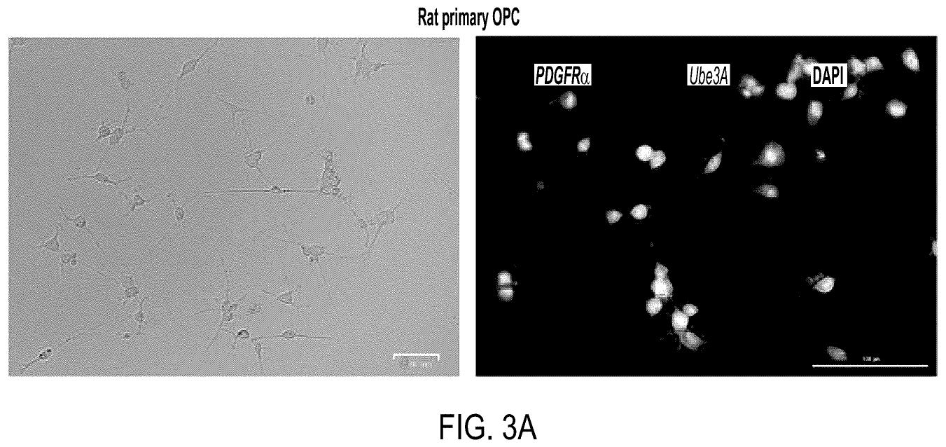

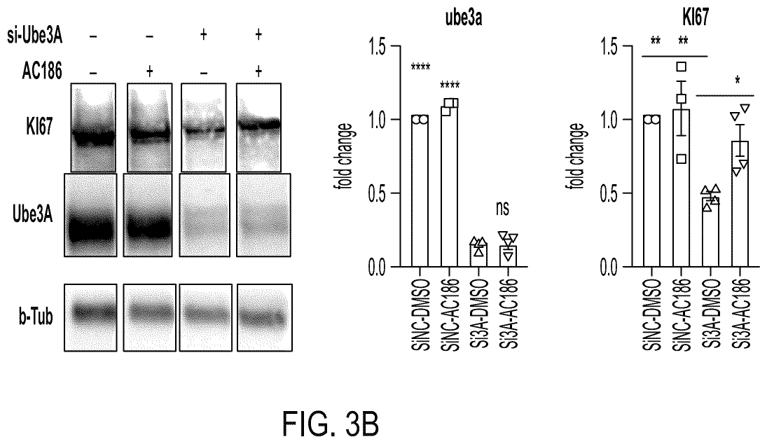

FIG. 3A shows the successful isolation of rat primary OPC cells with a light microscopy image. These cells expressed PDGFRa, and Ube3A, two OPC markers, as shown by fluorescence microscopy of stained cells. Ube3A was successfully knocked down with siRNA (FIG. 3B). FIG. 3B shows that RNAi-mediated Ube3A knockdown caused reduced OPC proliferation, which was rescued with the therapeutic compound AC-186.

### Example 8: Knockdown of Estrogen Receptor Beta in iOPCs Mimics Ube3A Knockdown

This example demonstrates that estrogen receptor beta is important for oligodendroglial functioning.

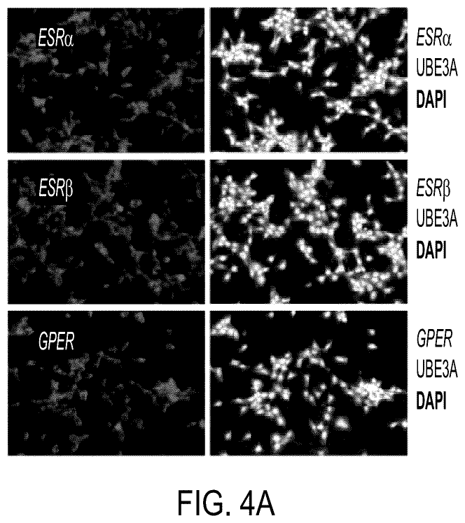

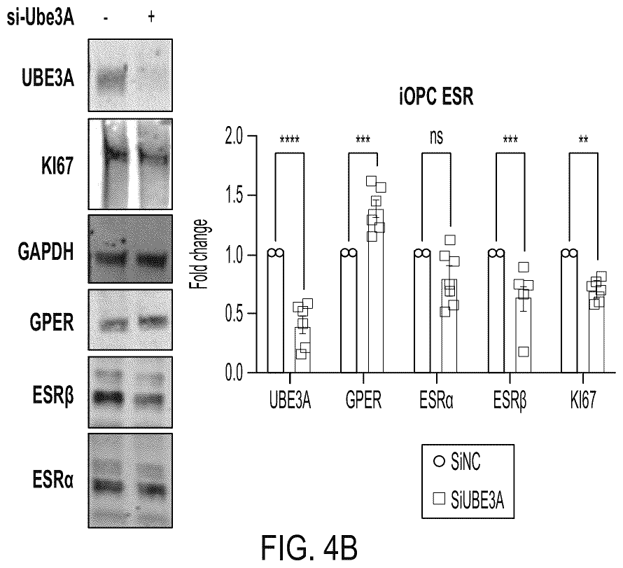

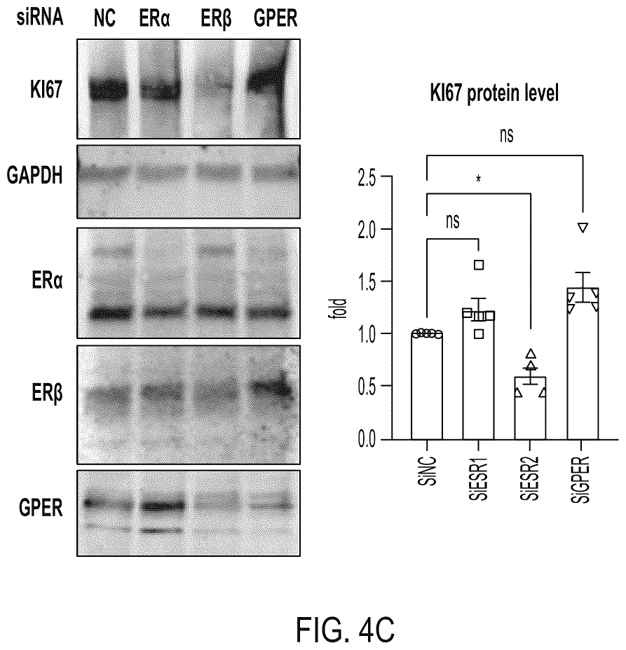

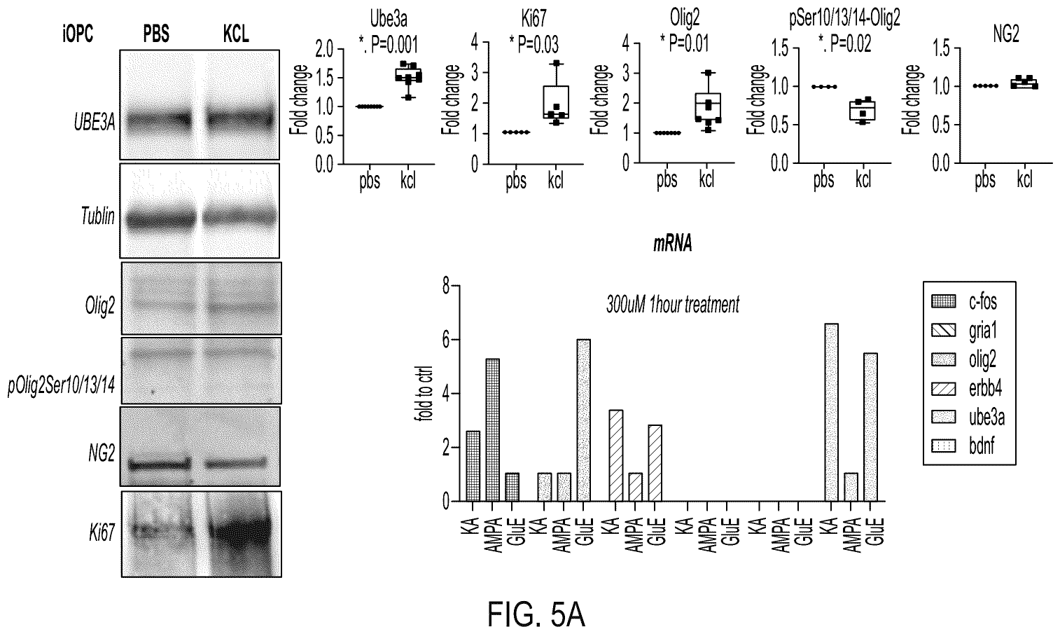

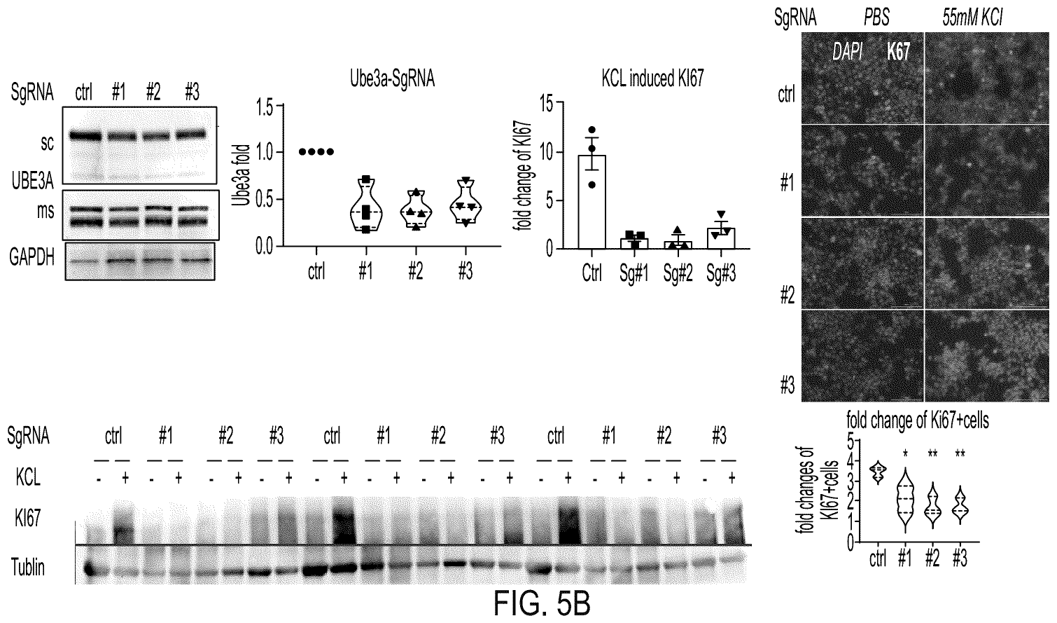

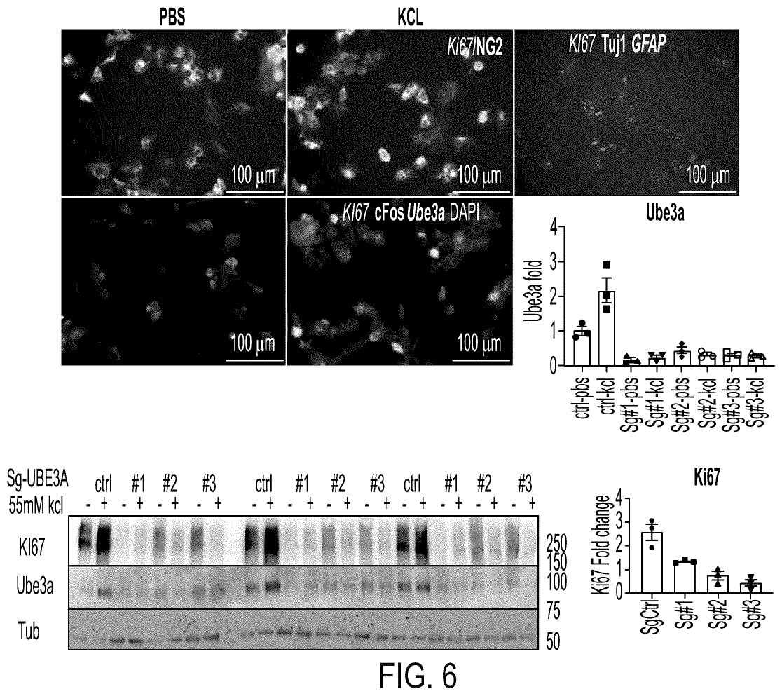

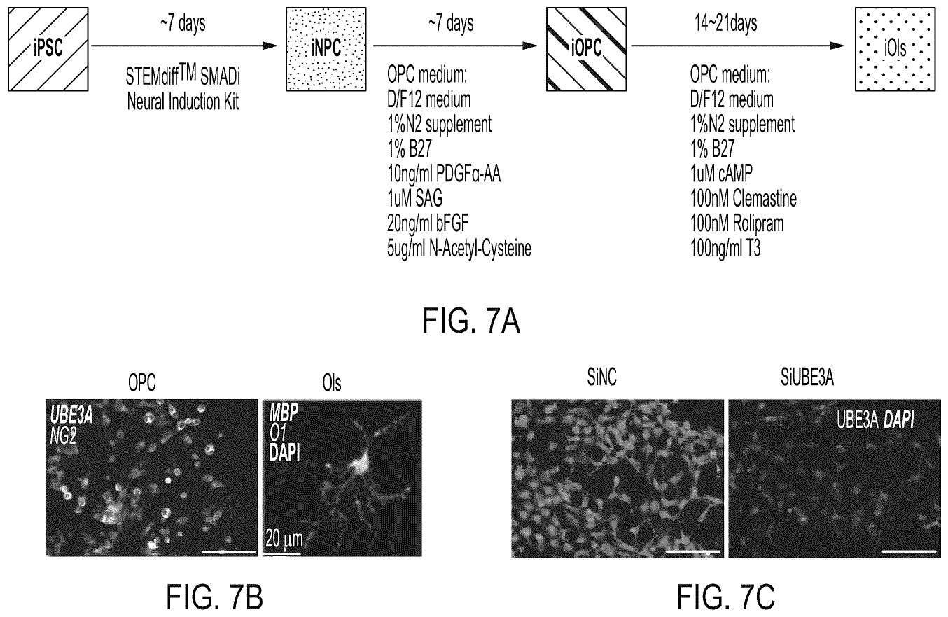

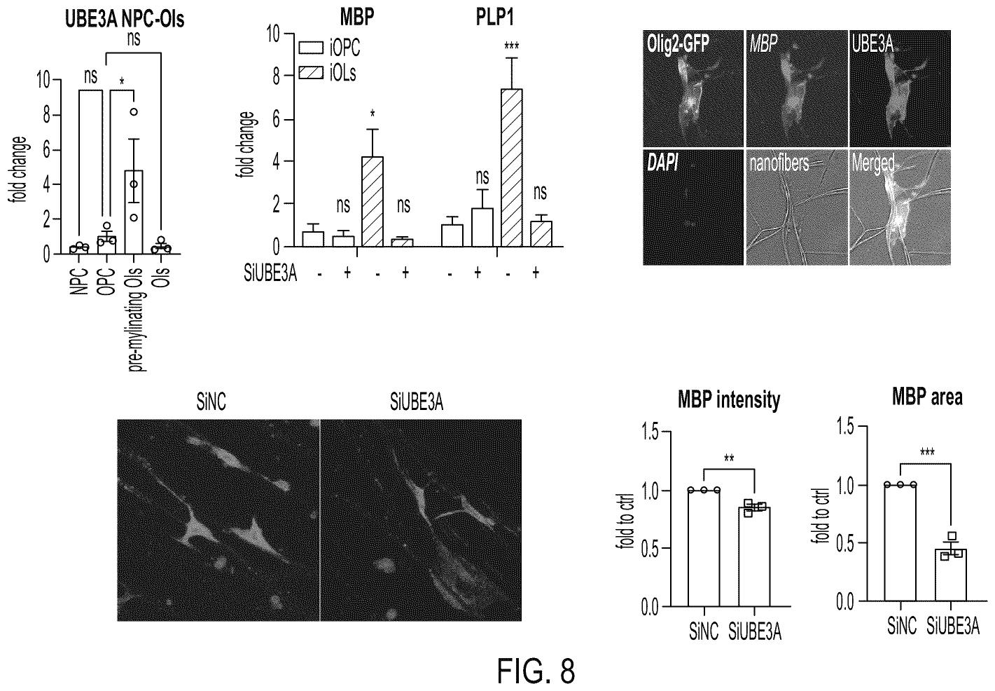

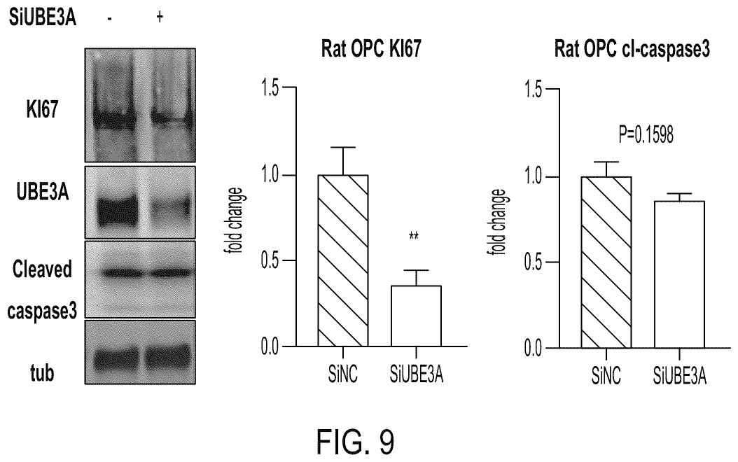

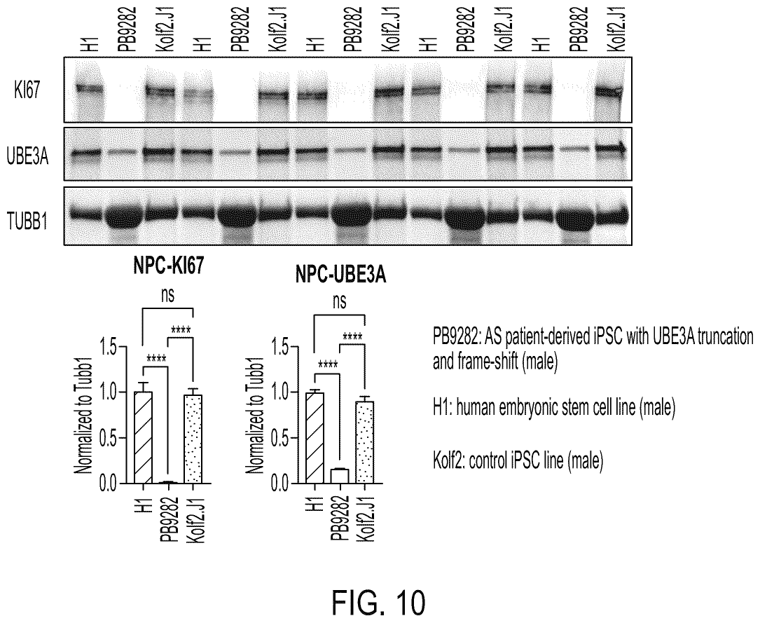

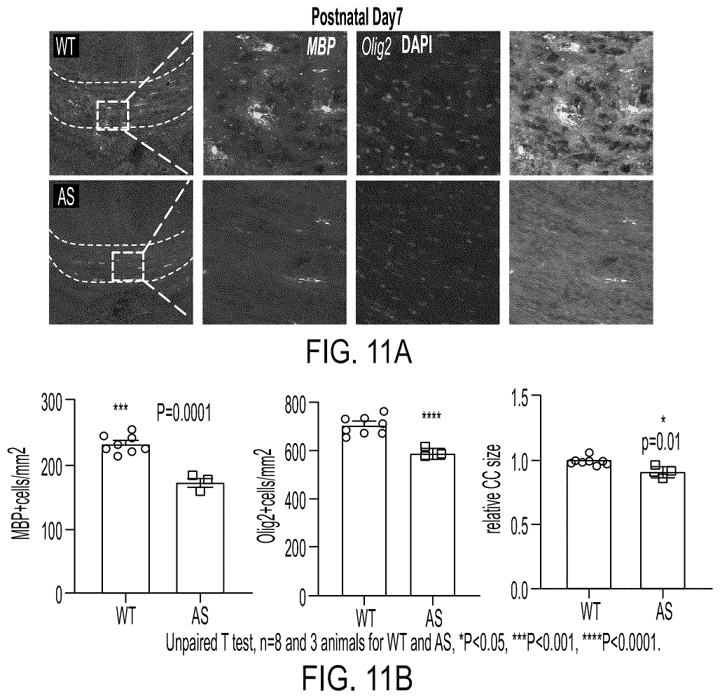

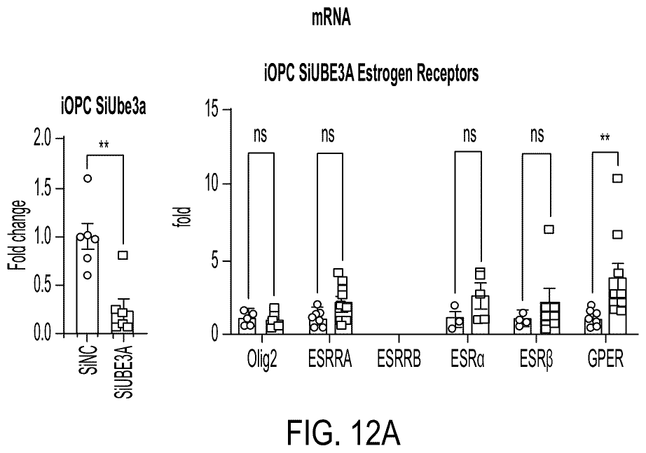

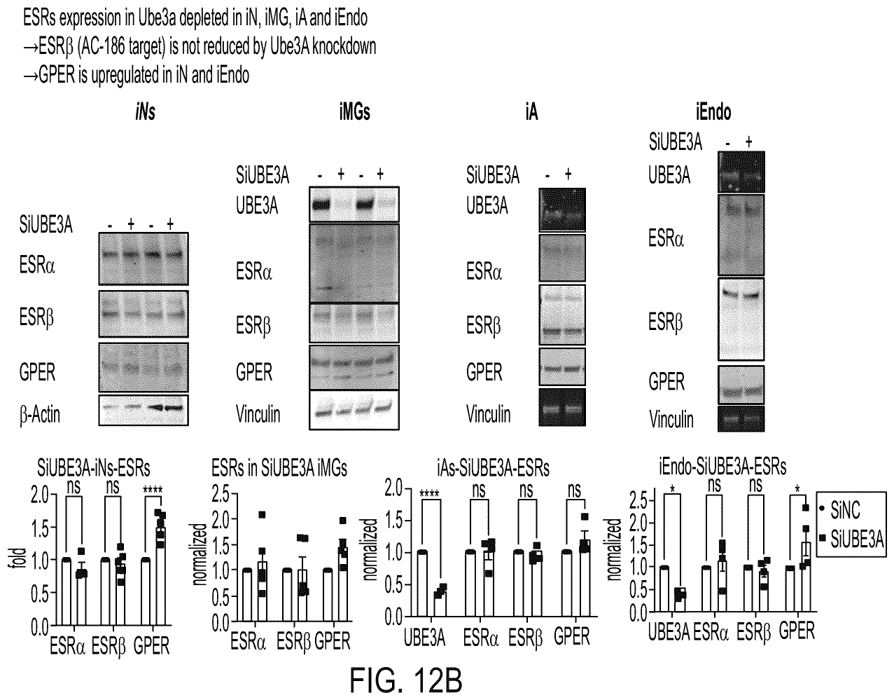

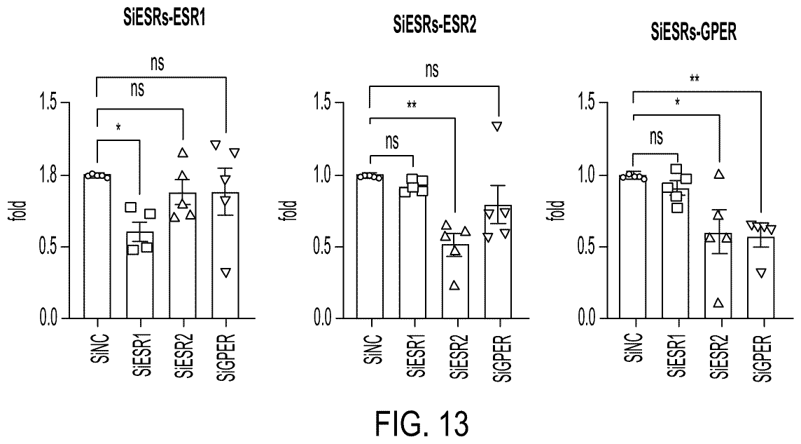

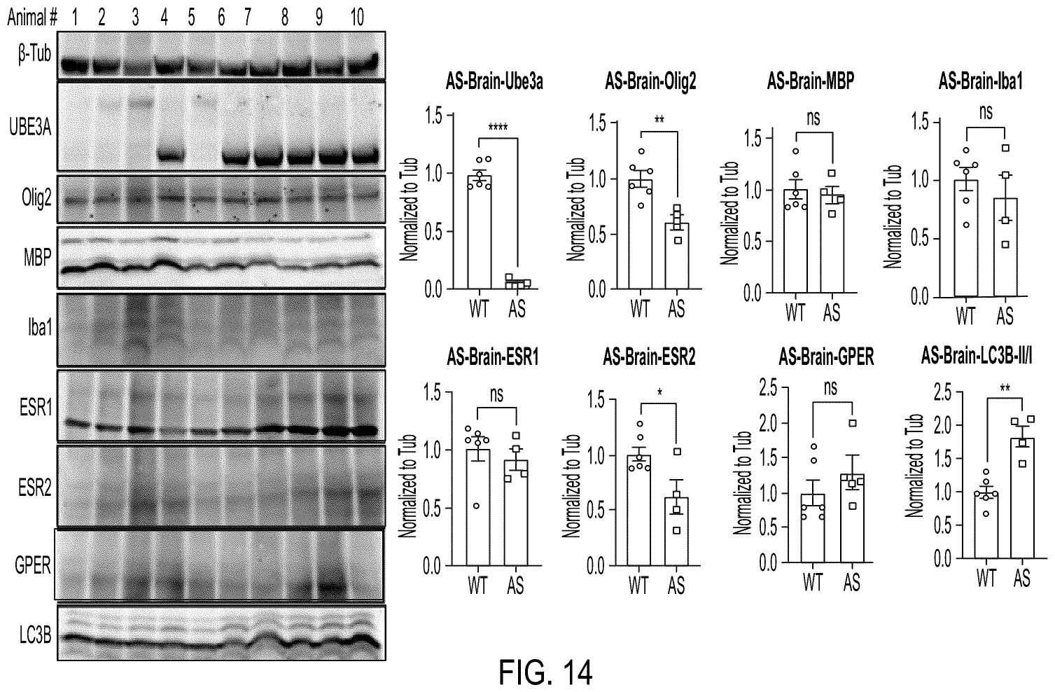

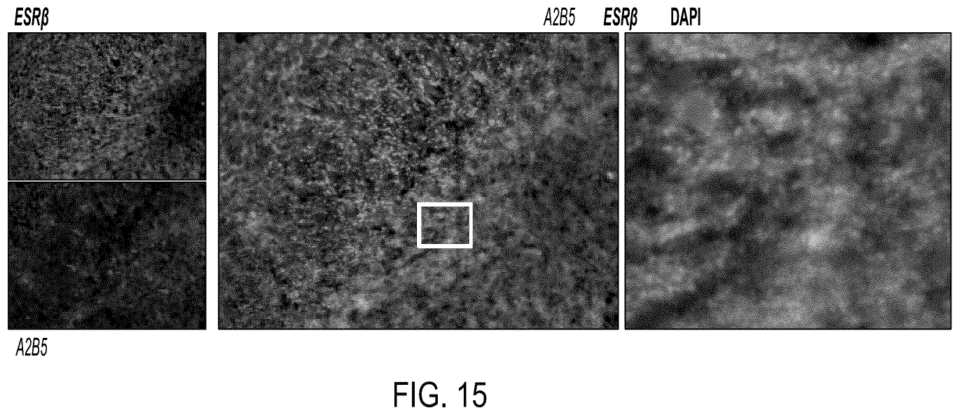

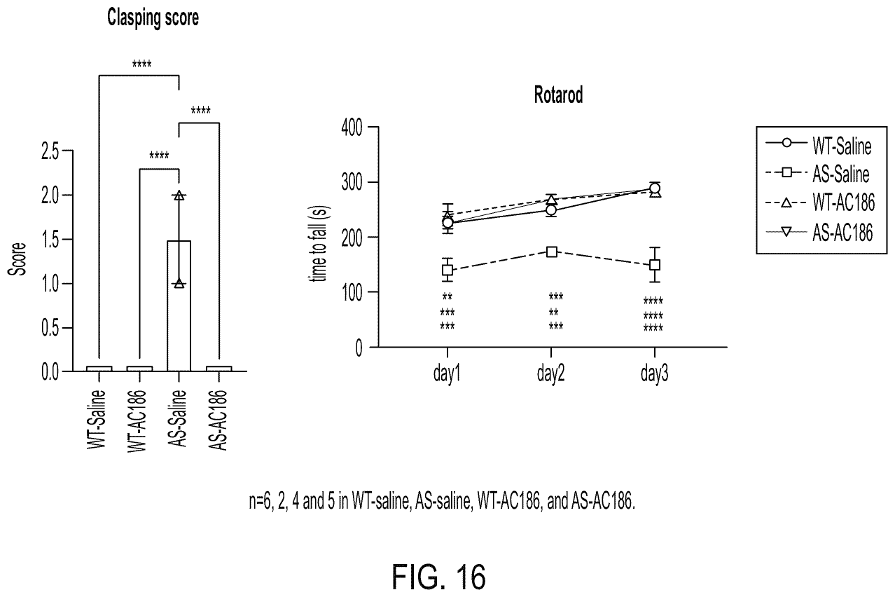

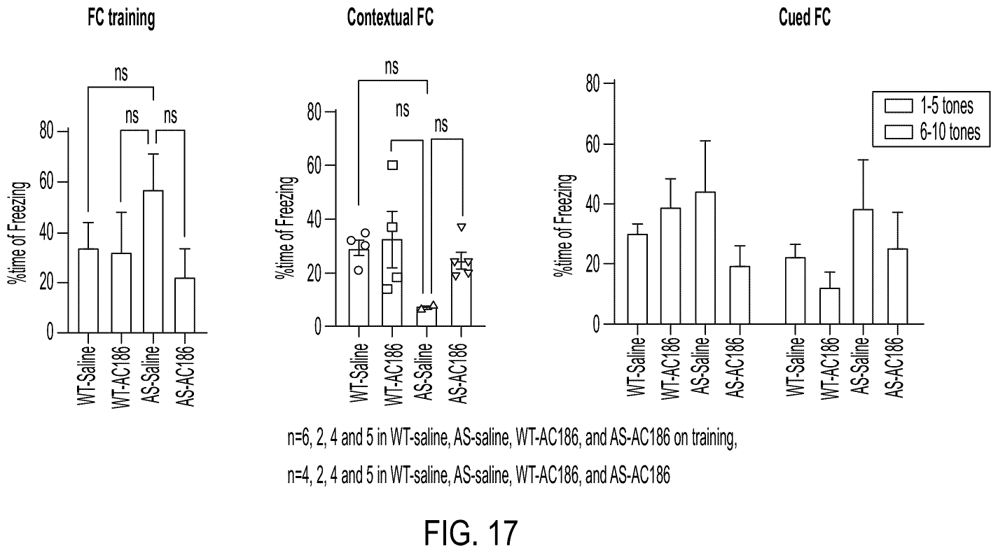

FIG. 4A shows that the following three receptors were expressed in iOPCs: estrogen receptor α (ERα), β (ERβ) and G-protein coupled ER (GPER). Further, FIG. 4B shows that Ube3A knockdown by siRNA upregulates GPER but downregulates ERβ, the selective target of AC-186 (FIG. 4B). This ERβ downregulation appears to be cell type-specific, as it is not observed in human iPSC-derived neurons, astrocytes, microglia or endothelial cells deficient of Ube3A (data not shown). The activation of estrogen receptors, either by the ligands (e.g. estrogen or AC-186) or by a ligand-independent mechanism involving cofactors, has been documented to regulate cell proliferation (see, e.g., Tremblay, G. B., et al. “Ligand dependent activation of the estrogen receptors alpha and beta by mutations of a conserved tyrosine can be abolished by antiestrogens” Caner Res 1998, Carascossa, S. et al. “CARMI mediates the ligand independent and tamoxifen resistant activation of the estrogen receptor alpha by cAMP” Genes Dev 2010, and Mal, R. et al. “Estrogen receptor beta (ERβ): A ligand activated tumor suppressor” Front Oncol 2020). Individual ERs in iOPCs were knocked down with siRNA. FIG. 4C shows that depletion of ERβ, but not ERα or GPER, significantly reduces cell proliferation and mimics Ube3A depletion. This finding suggests that, in AS, the expression and downstream signaling of ERβ is impaired in oligodendroglia.

### Example 9: The Therapeutic Efficacy of the ER Agonist AC-186 in AS Mouse Model

There is a downsized OPC population and deficient myelination in AS mouse brains up to at least 6 months of age. The oligodendroglial phenotypes will be longitudinally characterized at 5 timepoints from D7 to 9-12 months of age. A rescue study will then be performed by administering AC-186 at three timepoints (4-6 wks, 8-12 wks and 5-6 months of age). The robust behavioral assays of Open Field Test and Accelerating Rotarod (see, e.g., Sonzogni, M. et al “A behavioral test battery for mouse models of Angelman syndrome: a powerful tool for testing drugs and novel Ube3a mutants” Mol Autism 2018 and Huang, H. S., et al “Behavioral deficits in an Angelman syndrome model: effects of genetic background and age” Behav Brain Res 2013) will then be performed, followed by histopathological examinations of oligodendroglial phenotypes.

The oligodendroglial development, myelination and expression of ER in AS mice at will be chronically phenotyped at 5 timepoints from D7 to 9-12 months of age. Two standardized behavioral assays of Open Field Test and Accelerating Rotarod will be employed to validate and characterize the neuropsychiatric and motor learning defects in AS mice. The AC-186 treatment will then be administered at 3 timepoints before the behavioral assays. Tested animals will be sacrificed for detailed histopathological examinations of brain tissues in both AC-186 treated and control AS animals. An increase in myelination, OPC population, and/or OPC differentiation will indicate that the in vitro effects of AC-186 are recapitulated in vivo.

### Example 10: Characterization of the Oligodendroglial Dysfunction Along Development of AS Mouse Brains

The postnatal development of oligodendroglia will be further characterized with quantification of ER expression and myelination formation throughout the adulthood at 5 timepoints: D7, 4-6 weeks, 8-12 weeks, 5-6 months and 9-12 months. The mouse brains harvested at the indicated ages will be coronally sectioned for immunohistochemical staining with Ube3A, together with the markers of OPCs proliferation (NG2, Olig2, PDGFα or A2B5), differentiation (O4), and oligodendrocyte maturation (O1 and MBP). A standard myelin staining kit will be used in tissues older than 4 weeks to characterize the developmental myelination. Multiple brains regions will be analyzed, including corpus callosum, frontal cortex, hippocampus and cerebellum.

### Example 11: Investigation the Behavior Phenotypes of Neuropsychiatric and Learning Defects in AS Mice

Two standardized behavioral assays of Open Field Test and Accelerating Rotarod will be used to test the 2 most commonly described phenotypes in AS, the anxiety-like behaviors and the motor learning defect, respectively. These 2 assays will be performed on AS mice at 3 ages: 4-6 weeks, 8-12 weeks and 5-6 months. To test locomotor activity and anxiety, the Open Field Test will be carried out similarly as described in the literature, with a 110-cm-diameter circular open field and the light intensity at the center monitored and controlled. The total travel distance and time spent in different zones (defined by the radius) will be recorded by an infrared camera (Noldus® Wageningen, NL) and analyzed by the Etho Vision® software (Noldus® Wageningen, NL). To assess the balance and motor learning in coordination, the Accelerating Rotarod test will be performed with the standardized instrument (Ugo-Basile, Stoelting Co., Wood Dale, Il), with the settings of revolutions per minute (rpm; from 3-30), trial lengths and intervals as described in prior studies. The latency to fall, or to rotate off the top of the turning barrel will be measured by the build-in timer.

### Example 12: Rescue by AC-186 of Oligodendroglial and Behavioral Phenotypes in AS Mice

AC-186 has been demonstrated to have satisfactory bioavailability in multiple model organisms, including rodents and dogs (see, e.g., McFarland, K. et al. “AC-186, a selective nonsteroidal estrogen receptor beta agonist, shows gender specific neuroprotection in a Parkinson's disease rat model” ACS Chem Neurosci 2013). Mice will be treated at the age of 4 weeks, 8 weeks and 5 months with AC-186 or vehicle control DMSO through timed intraperitoneal (IP) injections, at a dosage of 10-30 mg/kg daily for 2 weeks. After the completion of AC-186 or DMSO treatment, the 2 behavioral assays will be performed as described in Example 11, followed by the histological examinations for quantifications of oligodendroglial cell density and ER expression as well as the myelination status as described in Example 10 above.

### Example 13: UBE3A Controls Oligodendroglial Homeostasis Via Estrogen Receptor-β Signaling

This example describes a mechanism for oligodendroglial homeostasis controlled by UBE3A, an E3 ubiquitin ligase known for its multifaceted roles in brain development. Using human iPSC-derived OPCs for a compound screen, it was shown that reduced estrogen receptor-β signaling underlies the impaired self-renewal caused by UBE3A loss. A downstream signaling cascade with a role in OPC proliferation was also identified using patient-derived iPSCs and a mouse model of Angelman syndrome (AS) characterized by UBE3A loss. Finally, selective estrogen receptor-β agonist treatment restores both oligodendroglial homeostasis and behavioral functions. These findings highlight a UBE3A-dependent, cell autonomous pathway in OPC self-renewal, offering therapeutic avenues for disrupted oligodendroglial homeostasis in neurodevelopmental conditions like AS.

### Introduction

UBE3A, known as E6-associated protein (E6AP), is a multifunctional ubiquitin ligase implicated in both oncogenic processes and neurological disorders through various signaling pathways, some of which do not involve its ubiquitin ligase activity. UBE3A can regulate cell proliferation and plays a role in brain development. The functional loss of UBE3A is directly associated with Angelman syndrome (AS); but excess dosage of the UBE3A gene markedly increases the penetrance of autism spectrum disorders, suggesting that UBE3A expression level must be regulated during brain development. Notably, UBE3A is expressed in both neurons and oligodendroglia across human and mouse brains. In AS, both human patients and mouse models exhibit diminished oligodendroglial populations and compromised myelination.

This study investigates whether UBE3A can modulate OPC self-renewal and sustain oligodendroglial homeostasis via an intrinsic mechanism. Employing human iPSC-derived OPCs (iOPCs), UBE3A deficiency was demonstrated to lead to reduced OPC proliferation and subsequent myelination deficits, which occur independently of PDGFRa signaling. To elucidate the specific role of UBE3A in OPC proliferation, UBE3A-deficient iOPCs were used to conduct a drug screen targeting a broad array of cellular functions. Estrogen receptor-β (ESRβ) agonists were identified as effective in compensating for impaired OPC self-renewal, implying a dysfunction in ESRβ signaling. Utilizing both AS patient-derived iPSCs and an AS mouse model, the functional consequences of UBE3A loss were validated. A marked decrease in estrogen receptor-β expression and signaling in AS-derived OPCs was observed. Treatment with selective estrogen receptor-β agonists successfully restored oligodendroglial homeostasis and ameliorated learning behaviors. These results establish a UBE3A-dependent, cell-autonomous pathway with a role in OPC self-renewal, presenting viable therapeutic strategies for addressing disrupted oligodendroglial homeostasis in brain disorders such as AS.

### Results

### Cell-Autonomous Reduction of OPC Proliferation Due to UBE3A Loss Rescued by ESR-β Stimulation

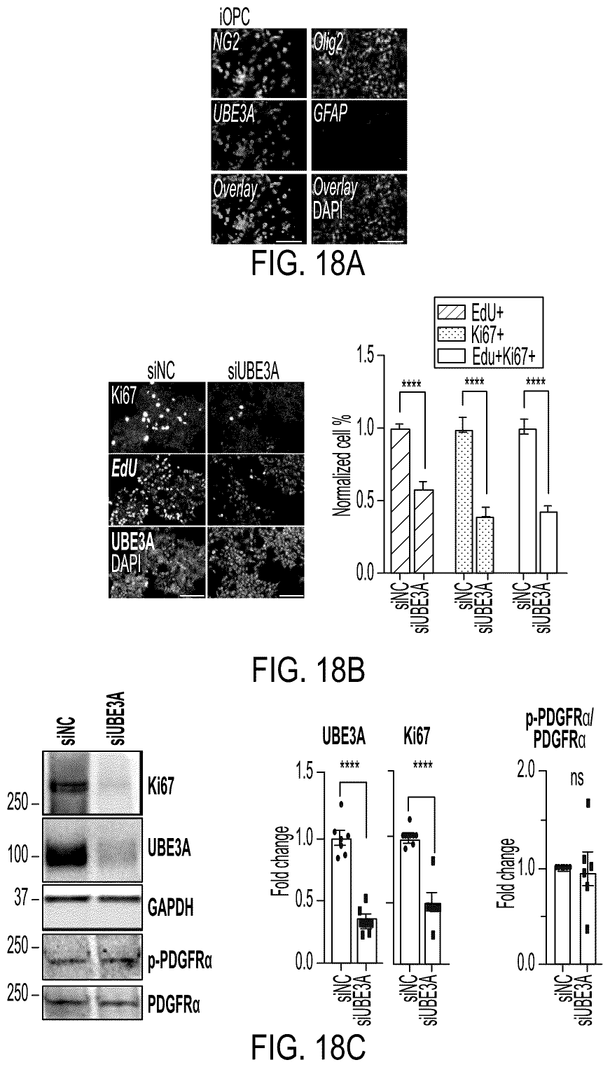

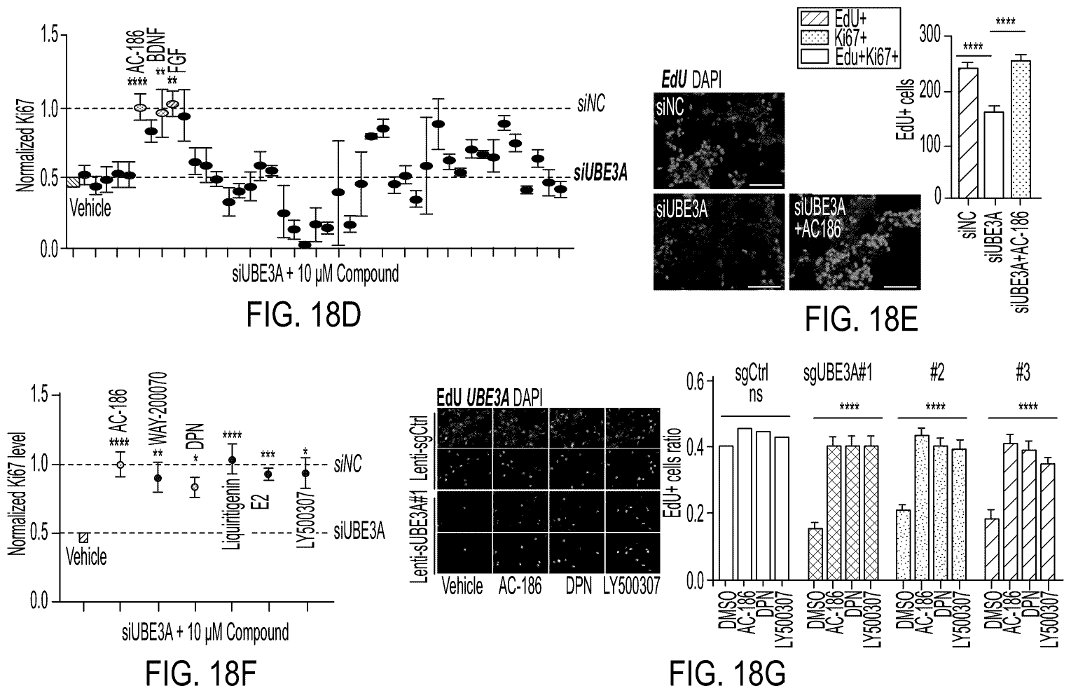

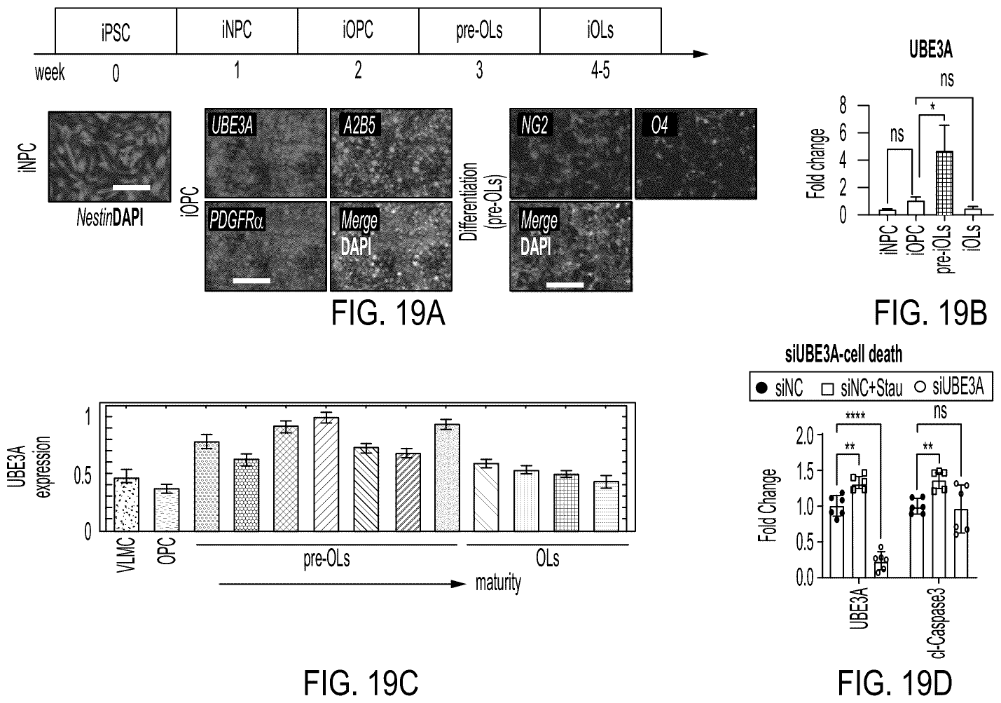

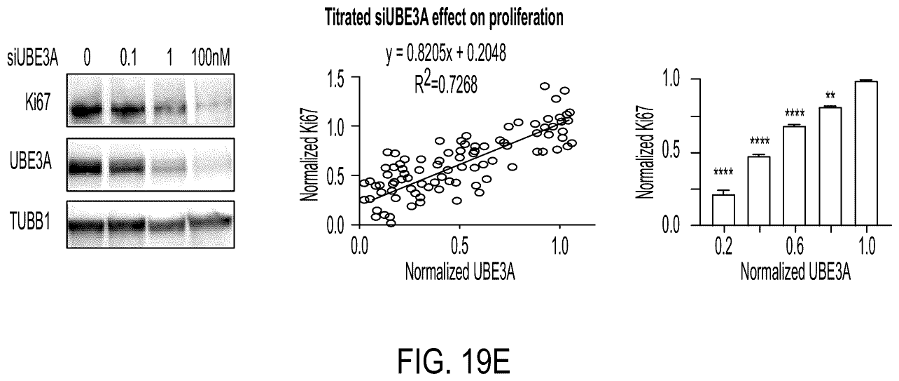

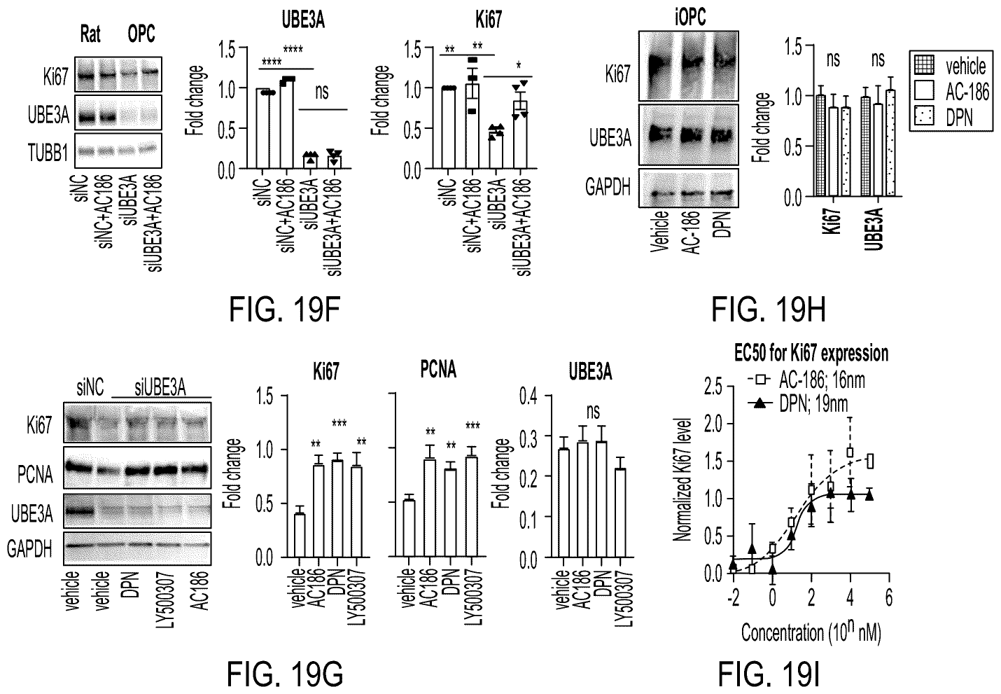

To investigate the influence of UBE3A on oligodendrocyte functions during brain development, human iPSC-derived oligodendrocyte lineage cells were utilized and adopted a chemically-defined protocol (FIG. 19A) to monitor oligodendroglial proliferation and differentiation from oligodendrocyte precursor cells (OPCs) through pre-myelinating oligodendrocytes (pre-OLs) to mature oligodendrocytes (OLs). This protocol facilitated an accelerated yet comprehensive evaluation across developmental stages, demonstrating proper morphology and specific marker expression (FIG. 18A, FIG. 19A). Importantly, UBE3A was consistently expressed across all stages, with levels particularly heightened in OPCs and pre-OLs (FIG. 19B), aligning with data from single-cell oligodendroglial transcriptomics of developing mouse brains (e.g., Marques et al. 2016, Science 352, 1326-1329) (FIG. 19C). The dynamic nature of OPCs, which continuously self-renew to maintain their population through local homeostatic proliferation, was further examined under conditions of UBE3A downregulation. By applying targeted siRNAs, robust reduction in UBE3A expression in iPSC-derived OPCs (iOPCs) was achieved, which significantly impaired their proliferation as evidenced by reductions in Ki67 expression and EdU incorporation (FIG. 18B-18C) but did not induce cell death (FIG. 19D). The proliferation impairment was not associated with a change in PDGFRa receptor activation (FIG. 18C). Further, a direct correlation between UBE3A protein levels and iOPC proliferation was established (FIG. 19E); even a moderate decrease in UBE3A (20-40%) significantly slowed iOPC proliferation.

Given the scarcity of knowledge regarding UBE3A function in oligodendrocyte biology, a comprehensive exploration of various cellular functions was performed, leveraging the high-throughput capabilities of the iPSC-based methodology (e.g., as described herein). We selected over 45 compounds with known targeted cellular actions (Table 1). These were chosen based on their documented effects on UBE3A, their ability to promote oligodendrocyte development and myelination, or their protective 10 roles in neural tissue. UBE3A expression was modulated using siRNA, achieving a controlled reduction that decreased OPC proliferation by approximately 50%, as evidenced by Ki67 protein levels. This setup allowed evaluation of the capability of each compound's ability to mitigate the proliferation deficits induced by UBE3A reduction, thereby revealing potential underlying cellular mechanisms (FIG. 18D).

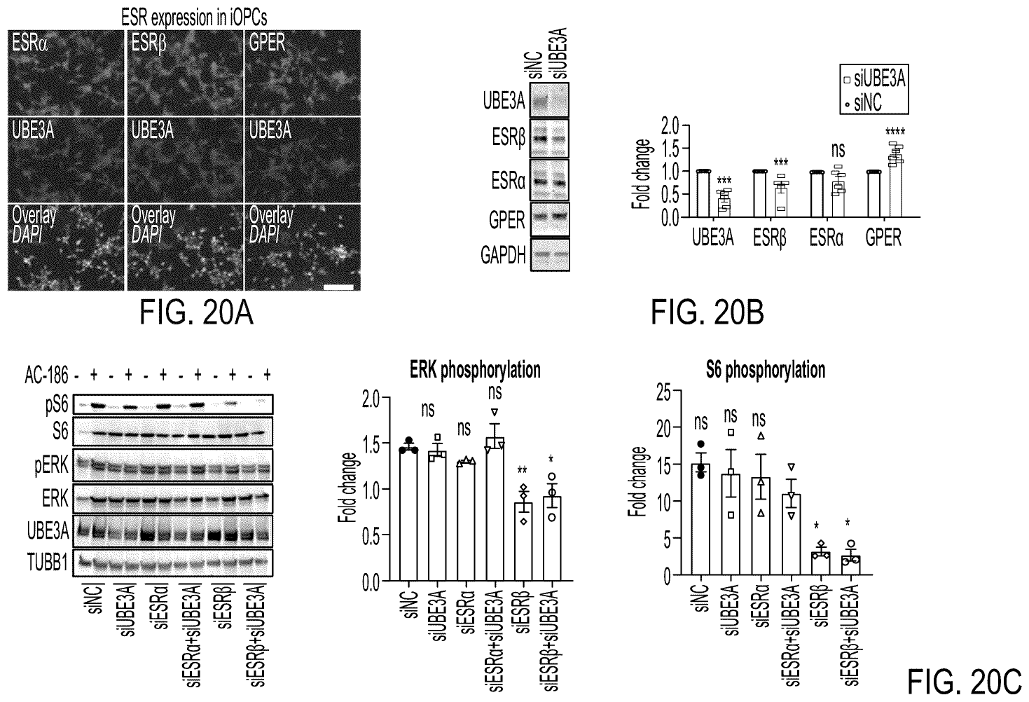

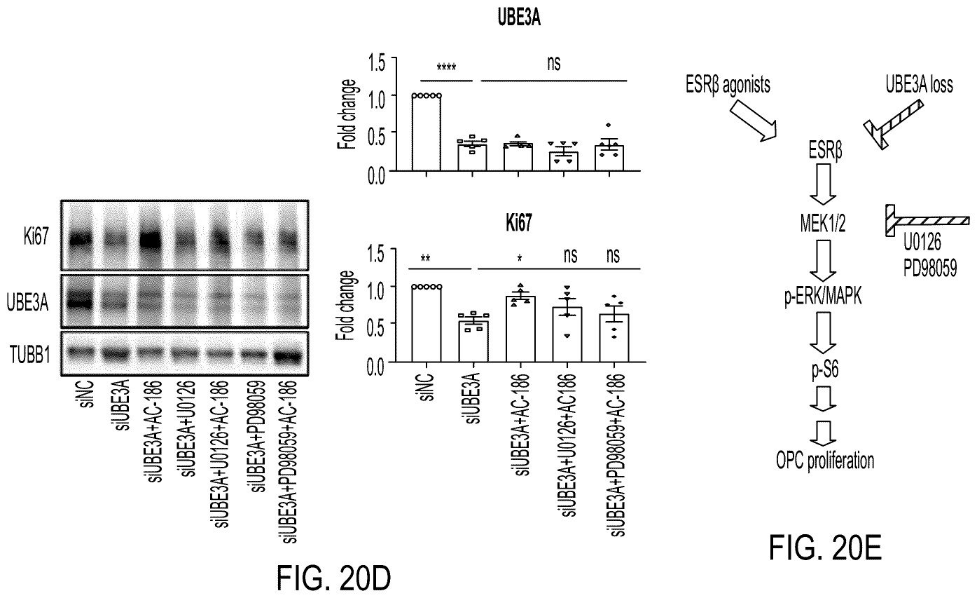

TABLE 1
Compounds selected for screening.

| | Compound name | Target cellular function | Catalog # |
|---|---|---|---|
| FIG. 20A | | | |
| 1 | DMSO | vehicle control | |
| 2 | Rolipram | selective phosphodiesterases PDE4 inhibitor | MedChem Express HY-16900 |
| 3 | Rapamycin | potent and specific mTOR inhibitor | Selleck Chemicals S1039 |
| 4 | Clemastine | histamine H1 receptor antagonist | MedChem Express HY-B0298A |
| 5 | Strychnine | strychnine-insensitive modulatory site of the NMDA | Millipore Sigma S0532-5G |
| | | receptor | |
| 6 | Ketoconazole | CYP3A4 and CYP24A1 inhibitor | MedChem Express HY-B0105 |
| 7 | AC-186 | estrogen receptor β (ERβ) agonist | Tocris #5053 |
| 8 | SHH, human recombinant | morphogen Sonic Hedgehog | MedChem Express HY-P70467 |
| 9 | BDNF, human recombinant | neurotrophin that belongs to NGF-beta family | STEMCELL Technologies 78005 |
| 10 | bFGF, human recombinant | fibroblast growth factor | R&D Systems, 233-FB-025/CF |
| 11 | PDGF-AA, human recombinant | platelet-derived growth factor, dimeric isoform AA | R&D Systems 221-AA-025 |
| 12 | Azathioprine | immunosuppressive agent | ThermoFisher J62314-03 |
| 13 | SUN11602 | aniline compound with basic fibroblast growth factor- | Selleck Chemicals S8192 |
| | | like activity | |
| 14 | Fingolimod | sphingosine 1-phosphate (S1P) antagonist | MedChem Express HY-12355 |
| 15 | FRAX1036 | PAK inhibitor | MedChem Express HY-19538 |
| 16 | Ezatiostat | glutathione S-transferase P1-1 (GSTP1) inhibitor | MedChem Express HY-13634A |
| 17 | Siponimod | sphingosine 1-phosphate (S1P) receptor modulator | MedChem Express HY-12355 |
| 18 | D-Syn3 | TrkB-PDZ binding inhibitor | (from Dr. John Marshall) |
| 19 | Tat-beclin 1 | inducer of autophagy | Selleck Chemicals S8595 |
| 20 | Indotecan | topoisomerase I inhibitor | HY-18351 |
| 21 | Acetylcholine chloride | neurotransmitter, a potent cholinergic agonist | MedChem Express HY-B0282 |
| 22 | 7,8-Dihydroxyflavone | TrkB agonist | MedChem Express HY-W013372 |
| 23 | GNF-5837 | pan-tropomyosin receptor kinase (TRK) inhibitor | MedChem Express HY-13491 |
| 24 | CHIR-99021 | GSK-3α/β inhibitor | MedChem Express HY-10182 |
| 25 | LM22A-4 | TrkB Agonist | MedChem Express HY-100673 |
| 26 | ANA-12 | TrkB antagonist | MedChem Express HY-12452 |
| 27 | BMP4, human recombinant | polymorphic ligand protein belonging to the TGF-β | R&D Systems 314-BP-010/CF |
| | | family | |
| 28 | Dorsomorphin | TP-competitive AMPK inhibitor | MedChem Express HY-13418A |
| 29 | CHI3L1 | secreted glycoprotein | OriGene Technologies TP303769 |
| 30 | 3,3′,5-Triiodo-L-thyronine | thyroid hormone receptors TRα and TRβ agonist | Fisher Scientific AAJ63312ME |
| | sodium | | |
| 31 | γ-Aminobutyric acid | ionotropic GABA receptors | MedChem Express HY-N0067 |
| 32 | SR-4370 | an inhibitor of HDAC | MedChem Express HY-111400 |
| 33 | Purmorphamine | smoothened/Smo receptor antagonist | MedChem Express HY-15108 |
| 34 | G-15 | G-protein-coupled estrogen receptor (GPER/GPR30) | MedChem Express HY-103449 |
| | | antagonist | |
| 35 | ITSA-1 | activator of histone deacetylase (HDAC) | MedChem Express HY-100508 |
| 36 | Fesoterodine (L-mandelate) | muscarinic receptor (mAChR) antagonist | MedChem Express HY-70053A |
| 37 | Potassium chloride | potassium supplement | Sigma-Aldrich P5405-250G |
| 38 | ABT-737 | potent Bcl-2, Bcl-xL, and Bcl-w inhibitor | MedChem Express HY-50907 |
| 39 | Forskolin | adenylate cyclase activator | Cayman 11018 |
| 40 | FGF17, human recombinant | fibroblast growth actor family member | ProSpec CYT-817 |
| 41 | G-1 | G-protein-coupled estrogen receptor (GPER/GPR30) | MedChem Express HY-107216 |
| | | antagonist | |
| 42 | CN-2097 | TrkB-PDZ binding | (from Dr. John Marshall) |
| 43 | TRO 19622 | mitochondrial-targeted neuroprotective compound | Tocris 2906 |
| 44 | Prostaglandin D2 | Prostaglandin | MedChem Express HY-101988 |
| 45 | 9-Aminoacridine | antimicrobial | Sigma-Aldrich A38401-5G |
| FIG. 20D | | | |
| 1 | DMSO | vehicle control | |
| 2 | AC-186 | estrogen receptor β (ERβ) agonist | Tocris #5053 |
| 3 | WAY-200070 | estrogen receptor β (ERβ) agonist | MedChem Express HY-101271 |
| 4 | DPN | non-steroidal estrogen receptor β (ERβ) selective | MedChem Express HY-12452 |
| | | ligand | |
| 5 | Estradiol | beta-estradiol | Millipore Sigma E4389-100MG |
| 6 | Liquiritigenin | estrogen receptor β (ERβ) agonist | MedChem Express HY-N0377 |
| 7 | Erteberel (LY500307) | estrogen receptor β (ERβ) agonist | ApexBio B1518 |

The screening identified specific compounds, notably brain-derived neurotrophic factor (BDNF), fibroblast growth factor (FGF) and AC-186, that effectively normalized proliferation levels in UBE3A-deficient iOPCs (FIG. 18D). AC-186 was particularly notable for its high specificity to ERβ. To rigorously validate our results across different platforms, primary OPCs were isolated from neonatal rat brains and replicated the reduction in proliferation by RNAi-mediated UBE3A depletion (FIG. 19F). Similarly, the proliferation inhibition was reversed by AC-186, as observed in human iOPCs (FIG. 18E). To ensure these effects were specific and not due to off-target actions, we compared AC-186 with other selective ERβ activators of different structural classes (Table 1), including WAY-200070. diarylpropionitrile (DPN). LY500307. Liquiritigenin, and estradiol (E2); all of which similarly mitigated the impact of UBE3A loss on OPC proliferation (FIG. 18F, 19G). Additionally, to address potential artifacts from siRNA-based knockdown, we utilized CRISPR/Cas9 technology to decrease UBE3A expression with multiple sgRNAs, confirming the consistent impact of UBE3A depletion on OPC proliferation and the therapeutic effects of ERβ agonists on impaired OPC proliferation (FIG. 18G). Notably, neither AC-186 nor DPN affected human or rat OPCs with normal UBE3A levels (FIG. 18F, 19H), highlighting their specific action in conditions associated with UBE3A deficiency. We determined effective concentrations (EC50) for AC-186 and DPN to restore Ki67 expression to be approximately 16 nM and 19 nM. respectively (FIG. 19I). This drug screening suggests a disruption in ERβ signaling as a likely pathological mechanism behind the oligodendrocyte dysfunction observed with UBE3A depletion.

### UBE3A Depletion Suppresses ESR-β Signaling Through MAPK/ERK Pathway in OPC Self-Renewal

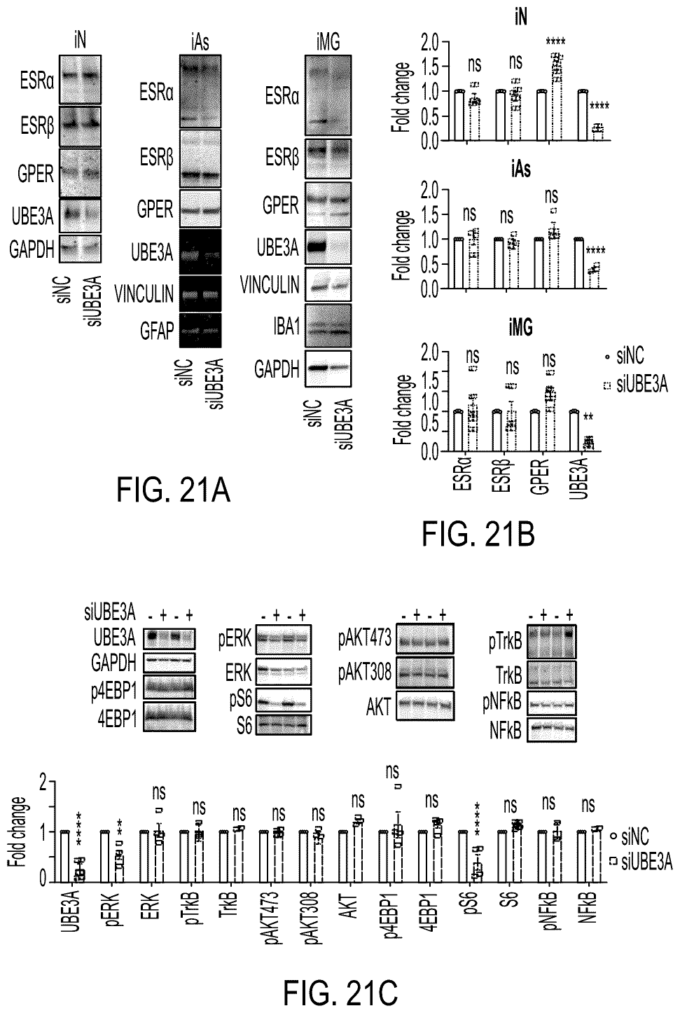

The role of estrogen receptor signaling in oligodendroglia was further explored. Estrogen receptors are prevalent in the brain, found in both neuronal and glial cells and are have a role in a variety of cellular functions independent of gender. This study confirmed that all three known receptors are expressed in iOPCs (FIG. 20A-20B): estrogen receptor-α (ESR-α), -β (ESR-β) and G protein-coupled estrogen receptor (GPER). The activation of estrogen receptors, either by ligands (e.g. estrogen or AC-186) or by a ligand-independent mechanism involving cofactors, is known to regulate cell proliferation by triggering well-characterized signaling pathways. Of note, UBE3A knockdown led to an increase in the expression of GPER and a concomitant decrease in the expression of ESR-β which is specifically targeted by AC-186 (FIG. 20B). Contrastingly, in the iPSC-derived human neurons. UBE3A knockdown resulted only in increased GPER levels without affecting ESR-α or ESR-β levels (FIG. 21A-21B). Furthermore, UBE3A depletion did not alter the expression of any estrogen receptors in our iPSC-derived astrocytes or microglia (FIGS. 21A-21B), suggesting a unique role in oligodendroglia of UBE3A depletion in ESR-β function.

Membrane-bound estrogen receptors (ESR-β and GPER) can also initiate rapid intracellular signaling cascades upon activation. The effects of UBE3A knockdown on multiple pathways downstream of estrogen receptor signaling, including mTOR/4EBP1, ERK/MAPK, PI3K/Akt, TrkB and NFkB, were examined in iOPCs. A reduction in the activation of the ERK/MAPK pathway and its phosphorylation target, S6, without significant changes in other pathways associated with ESR-β was found (FIG. 21C). Considering the role of the ERK/MAPK signaling pathway in the proliferation and differentiation of oligodendroglia, whether the loss of ESR-β was linked to the observed decrease in ERK/MAPK activity was tested. The UBE3A-depleted iOPCs were treated with the ESR-β agonist AC-186 and found that this treatment restored the activity of ERK and its downstream target, S6, while UBE3A levels remained unchanged (FIG. 20C). The beneficial effects of AC-186 on ERK/MAPK activation were specifically tied to ESR-β, as demonstrated by the fact that these effects were negated when ESR-β was knocked down. In contrast, knocking down ESR-α, which is structurally similar to ESR-β, did not reverse the effects of AC-186 (FIG. 20C). Moreover, the silencing of ESR-β, but not of ESR-α, replicated the consequences of UBE3A loss by diminishing activation in the ERK/MAPK pathway and S6 phosphorylation (FIG. 20C). This reinforces the specific involvement of ESR-β in the signaling dysfunction observed in oligodendroglia due to UBE3A deficiency. Additionally, when we introduced the highly selective MEK inhibitors, PD98059 and U0126, they inhibited the ERK/MAPK pathway activation stimulated by AC-186 and consequently reduced the effect of the compound to enhance the proliferation of iOPCs with UBE3A knockdown (FIG. 20D). These observations collectively showed that ESR-β is an activator of the ERK/MAPK signaling cascade in OPCs, which plays a role in regulating their proliferation (FIG. 20E). The disruption of this ESR-β/ERK pathway due to UBE3A depletion could potentially be corrected by the administration of an ESR-β agonist.

ESR-β Activation Mitigates Oligodendroglial and Behavioral Deficits Resulting from UBE3A Depletion in AS Mice.

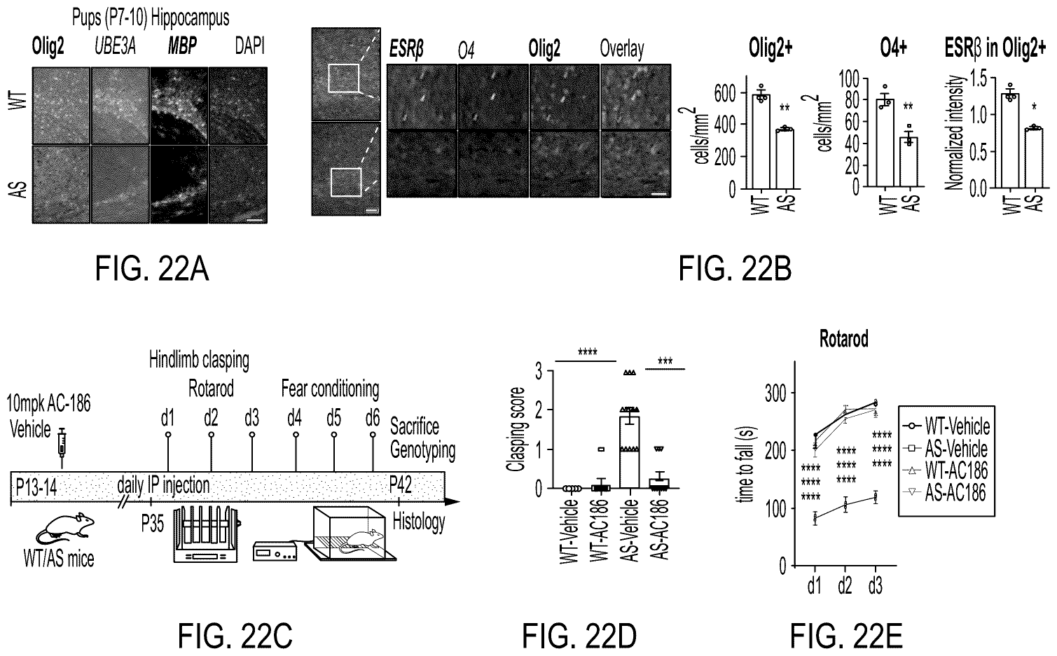

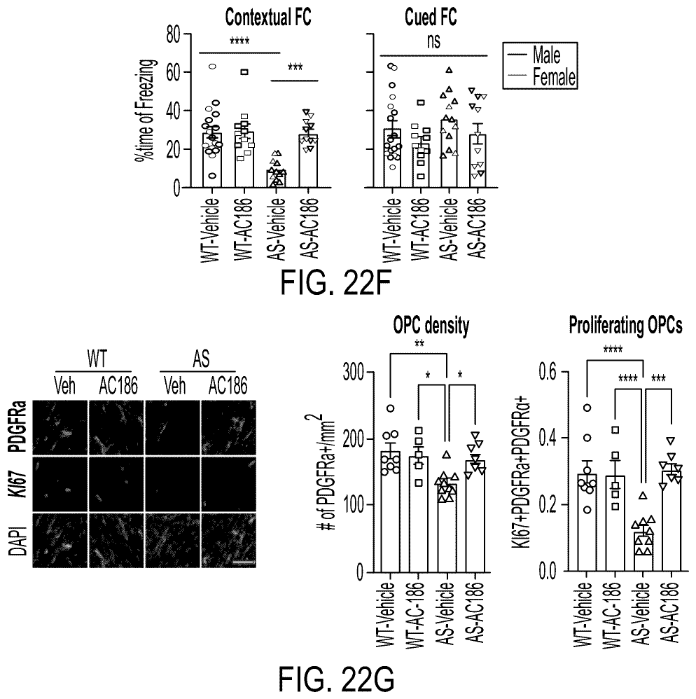

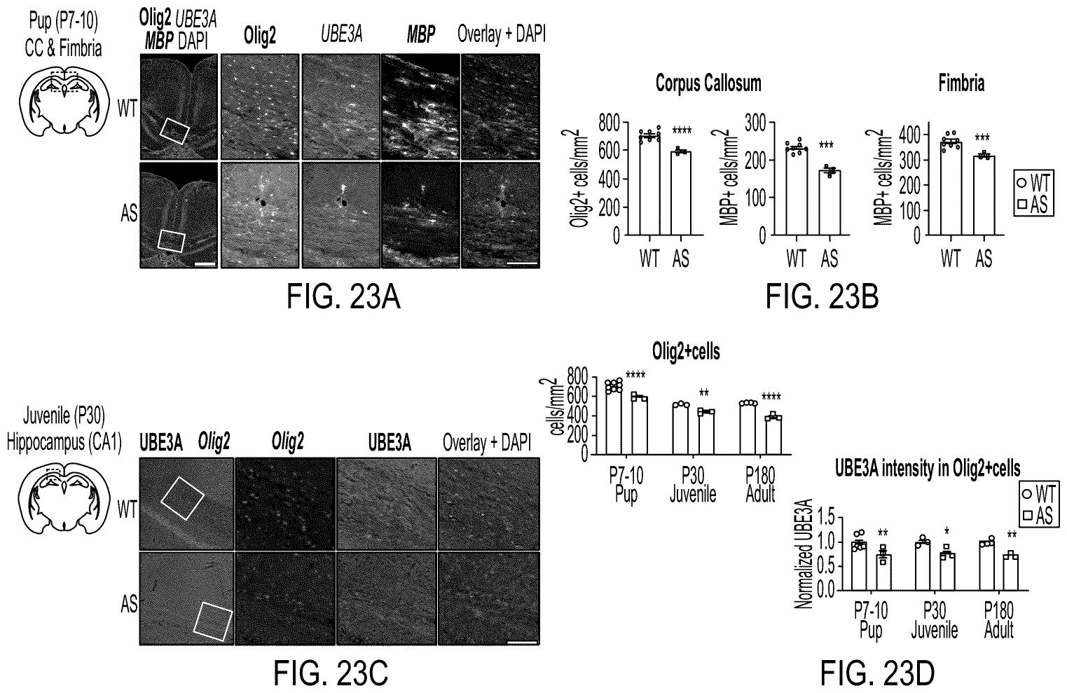

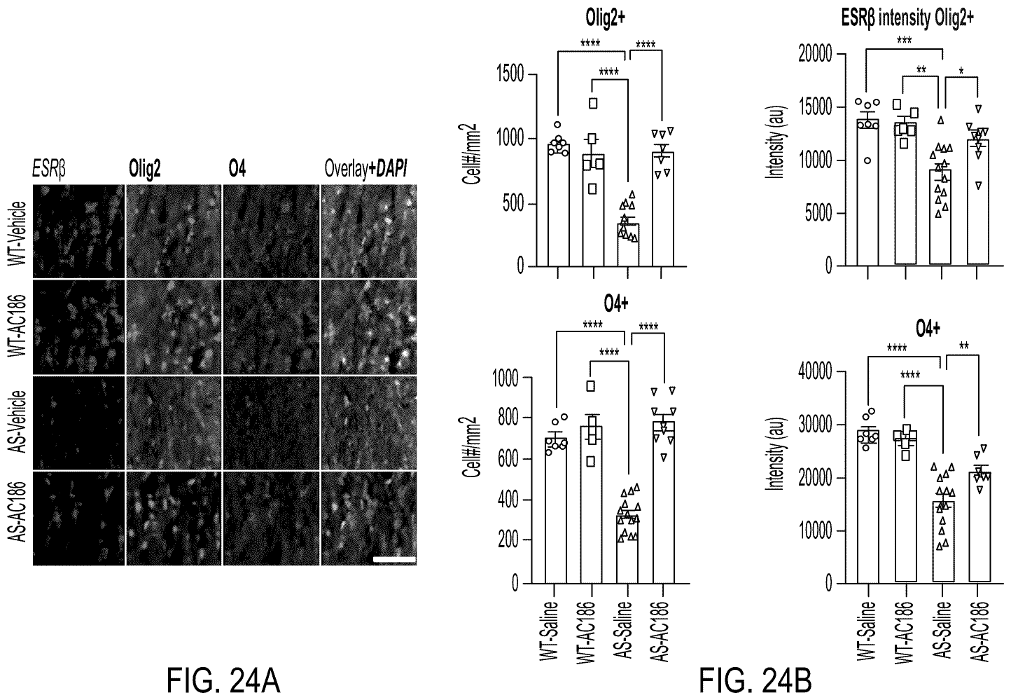

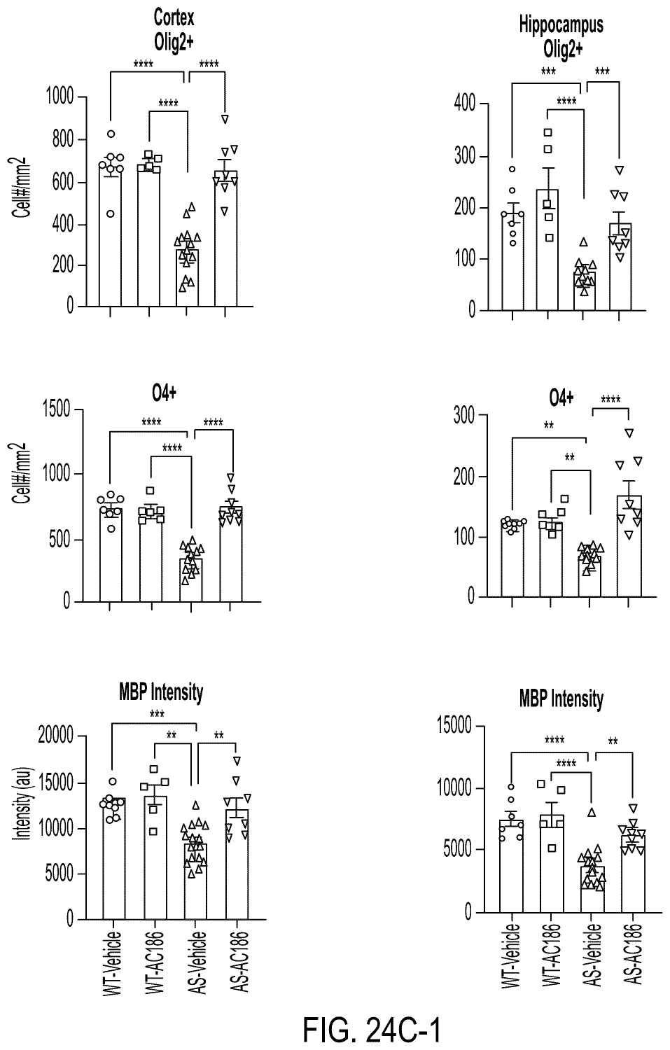

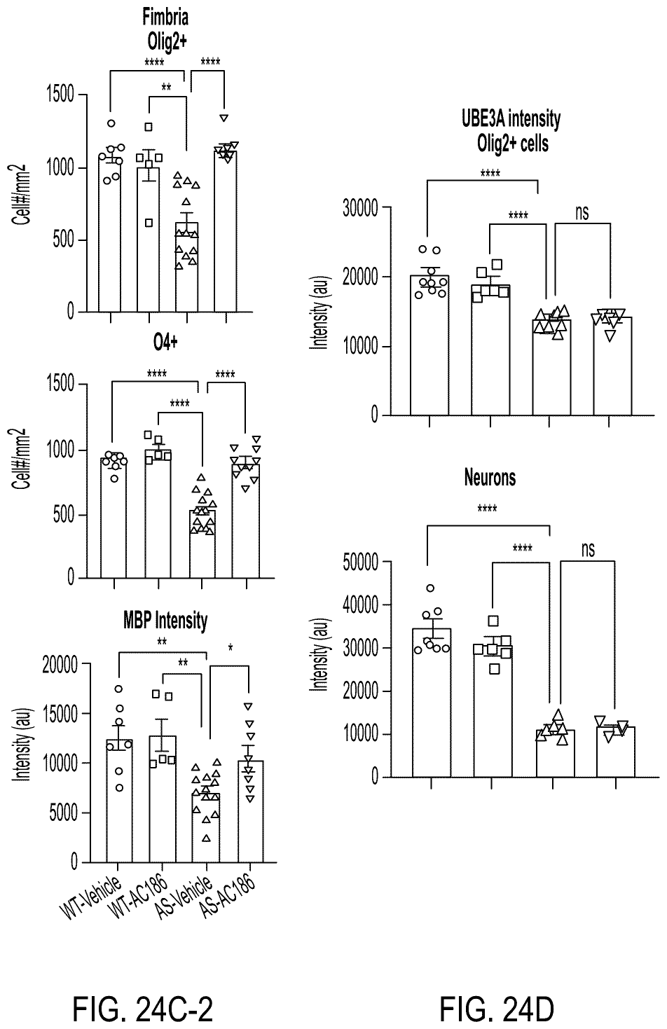

Whether UBE3A depletion corresponds to the oligodendroglial dysfunction in vivo, as observed in the heterozygous deletion model of AS mice, the UBE3A^{m-/p+} (or E6-AP UBE3A^{tm1Alb}) strain which recapitulates pathological developmental features including microcephaly, oligodendrocyte dysfunctions and behavioral abnormalities, was investigated. Homozygous UBE3A knockout mice exhibit more pronounced AS-related phenotypes, substantiating the role of proper UBE3A dosage in brain development. To assess the oligodendroglia population and myelination status across developmental stages, oligodendroglial marker expression in brain sections from developing AS mice during pup (P7-10), juvenile (P30), and adult (P180) stages was analyzed. AS pups exhibited downsized pools of oligodendrocyte lineage cells in the brain regions of the hippocampus (FIGS. 22A-22B) and the corpus callosum and fimbria of the fornix (FIGS. 23A-23B). In both juvenile and adult AS mouse brains, there was an intensified decline in oligodendrocyte populations (FIGS. 23C-23D). Notably, UBE3A expression was significantly reduced across all stages in AS mice compared to wildtype controls (FIGS. 22A, 23A, 23C-23D), suggesting that diminished UBE3A expression disrupts oligodendroglial homeostasis during neurodevelopment. To determine if UBE3A deficiency and the consequent reduction in ESRβ function observed in vitro could also be detected in the developing brains of AS mice, confocal microscopy studies were conducted to assess ESR-β colocalization with oligodendroglial markers (FIGS. 22B, 24A). A significant decrease in ESR-β expression in young AS mouse oligodendrocyte lineage cells was observed.

The restoration of oligodendroglial homeostasis via targeted augmentation of diminished ESR-β signaling as a potential therapeutic approach was explored. The ESR-agonist AC-186, which has effective bioavailability in various animal models including rodents and canines, was administered to both male and female juvenile AS mice (FIG. 22C). Established protocols of behavioral assays were used to assess drug efficacy, focusing on phenotypes characteristic of AS mice, such as deficits in contextual fear learning and motor learning impairments measured by the accelerating rotarod test. Additionally, the Hindlimb Clasping test, an observational measure indicative of CNS pathology, was employed. The Hindlimb Clasping test effectively differentiated AS mice from wildtype controls, providing a reliable phenotypic indicator (FIG. 22D). Notably, AC-186 treatment significantly alleviated neurological symptoms in AS mice, enhancing their performance to wildtype levels without affecting wildtype mice (FIG. 22D). The three-day Accelerating Rotarod protocol highlighted baseline motor learning impairments in AS mice and revealed significant improvements in AS mice treated with AC-186 compared to those receiving a vehicle solution (FIG. 22E). In the contextual fear conditioning test, juvenile AS mice showed pronounced deficits, which were ameliorated by AC-186 treatment, with no difference between genders (FIG. 22F). Conversely, the cued fear conditioning test showed no intergroup differences, suggesting this aspect of fear memory remains unaffected by the syndrome (FIG. 22F).

After conducting behavioral assessments, detailed histopathological examinations were used to evaluate the structural integrity and functional state of oligodendrocyte lineage cells, particularly OPCs and myelin production, alongside the expression of ESR-β in certain brain areas. The primary analyses centered on OPCs due to their dynamic responsiveness and sensitivity to genetic and environmental stresses in disease conditions. Prior to the administration of AC-186, a notable reduction in OPC density was observed across various brain regions of AS mice compared to wildtype controls. (FIG. 22G). This reduction was quantitatively assessed by counting dividing OPCs, revealing a more pronounced decrease (FIG. 22G). Further investigations revealed a decrease in both immature and mature oligodendrocyte lineage cells across gray and white matter regions, marked by specific developmental stage markers, indicating a decline in their populations as well as a deficiency in myelination within AS mouse brains (FIG. 24A-24D). These findings confirm a disruption in the homeostatic proliferation of OPCs in AS, impacting both the maintenance of OPC pools and the availability of precursor cells necessary for differentiation into myelinating oligodendrocytes. AC-186 had no impact on wildtype mice, and it notably restored oligodendrocyte populations and myelination in AS mouse brains (FIG. 22G, 24A-24C). Notably. AC-186 slightly increased ESR-β expression in AS oligodendroglia (FIG. 24B), but did not modify UBE3A levels (FIG. 24C). Overall, these data show that the activation of ESR-β could be an effective treatment strategy for disrupted OPC homeostasis.

### ESR-β Downregulation by UBE3A Deficiency Affects OPC Self-Renewal but not Myelination in Oligodendrocytes.

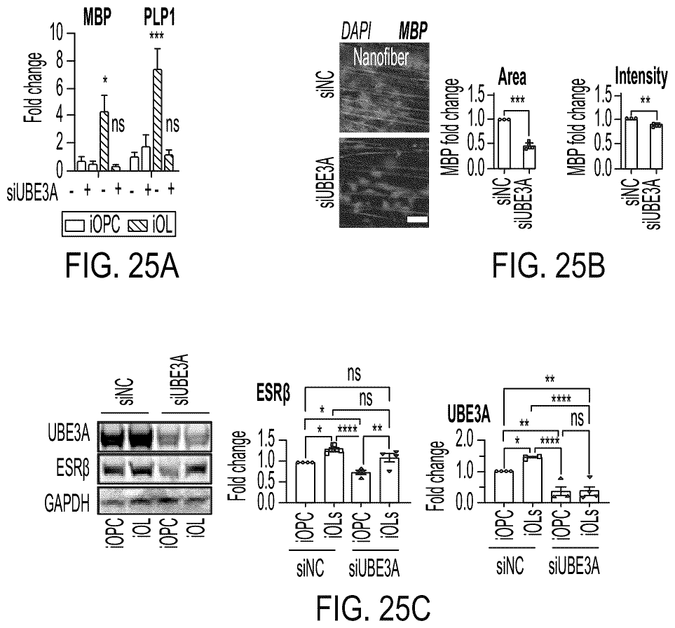

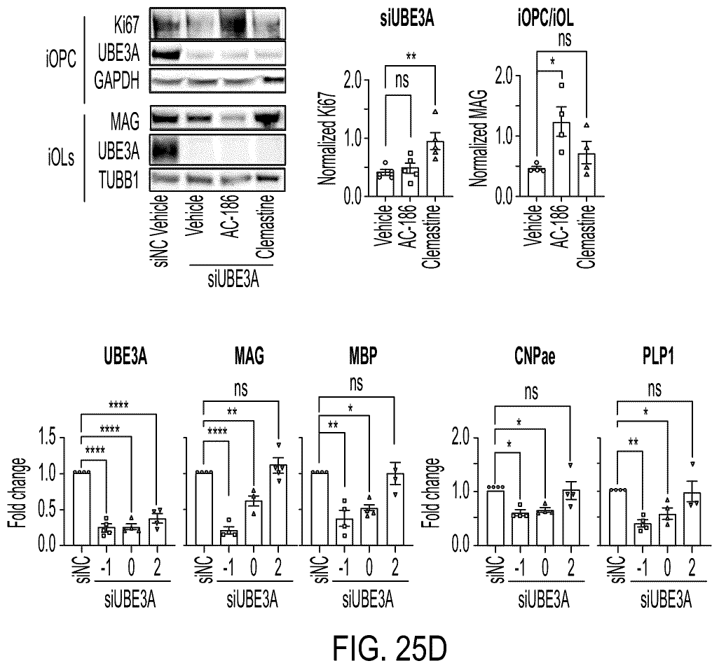

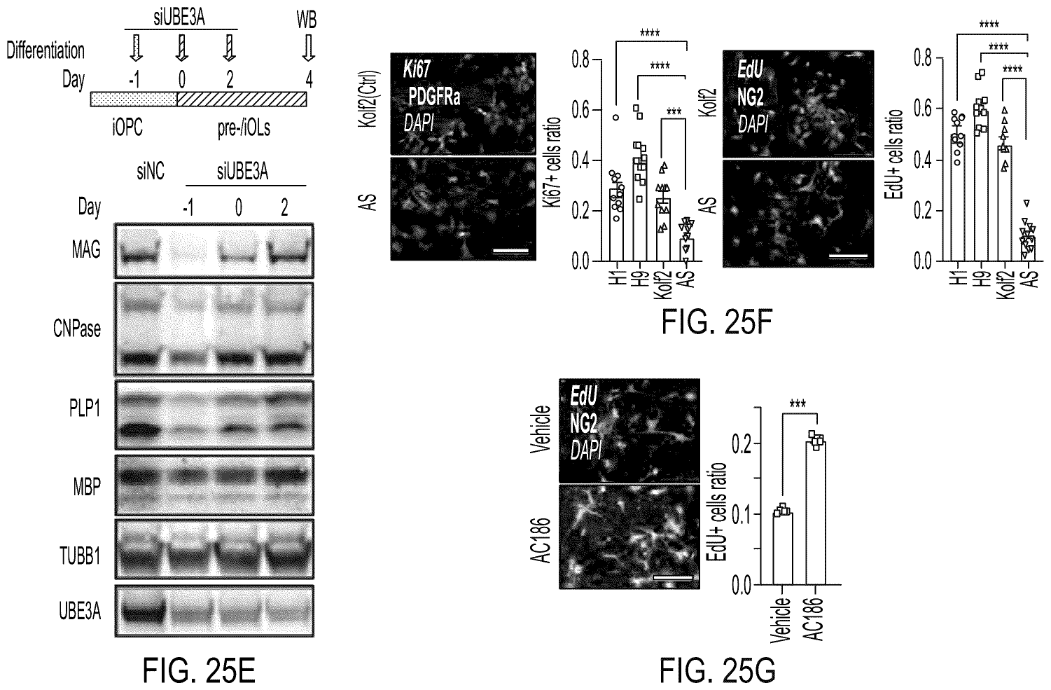

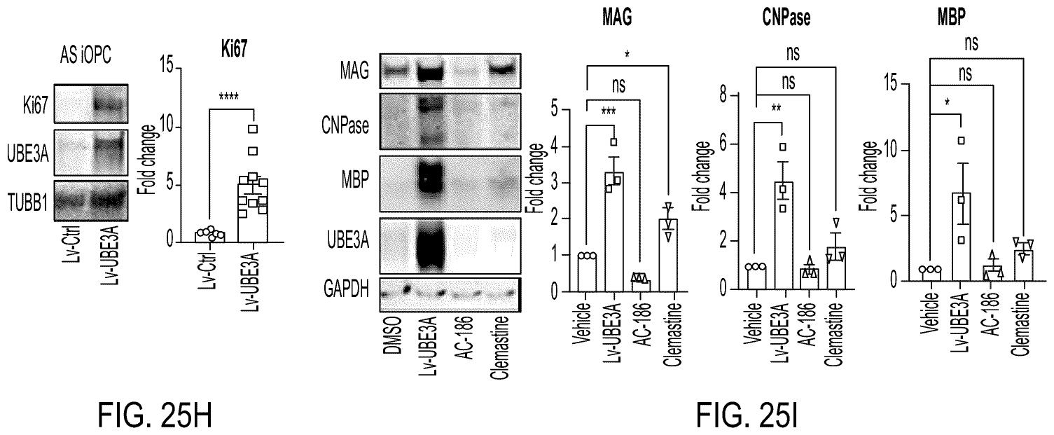

These in vitro and in vivo studies have jointly underscored a role for UBE3A in maintaining oligodendroglial homeostasis by regulating cell proliferation via ESR-β signaling. However, the influence of UBE3A on oligodendrocyte differentiation and maturation remained uncertain. The above results in mouse brains indicated that targeting the ESR-β pathway not only restored OPC density but also ameliorated the myelin deficiency resulting from UBE3A loss. To delve deeper into the effects of UBE3A depletion on oligodendrocyte development, iOPCs were differentiated into pre-myelinating (pre-iOL) and fully mature oligodendrocytes (iOL) (FIG. 19A, 26A-26B), with persistent UBE3A depletion. This differentiation resulted in a reduced population of mature oligodendrocytes, evidenced by decreased expression of mature oligodendrocyte markers (FIG. 25A) and impaired myelination capacity, particularly in their ability to ensheath synthetic nanofibers with myelin (FIG. 25B). The downregulation of ESR-β induced by UBE3A depletion was not observed after the differentiation of iOPCs into iOLs (FIG. 25C), highlighting a major shift in cellular state.

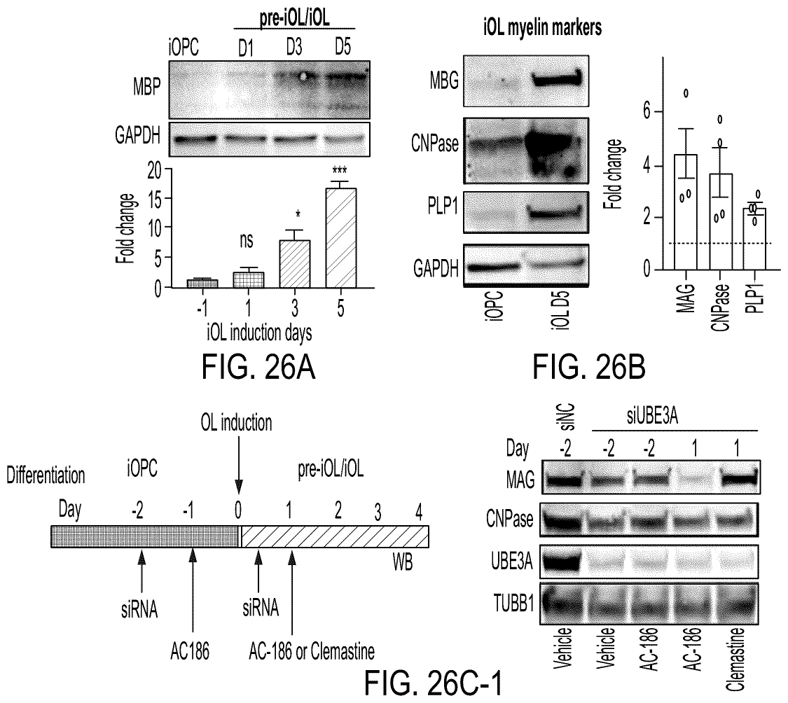

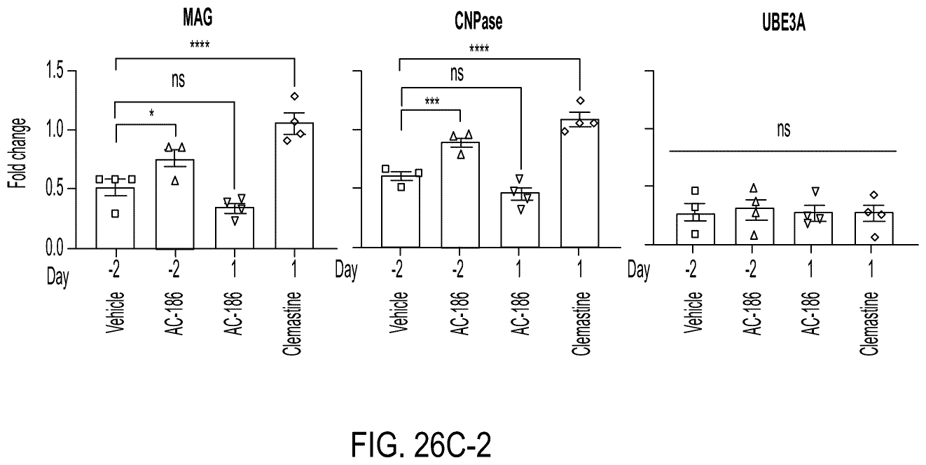

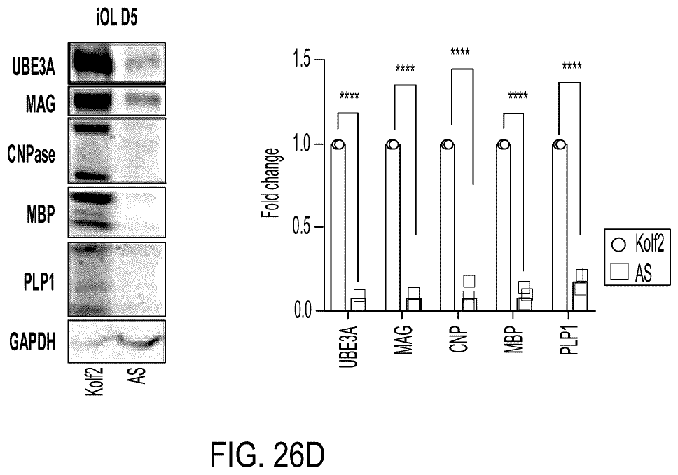

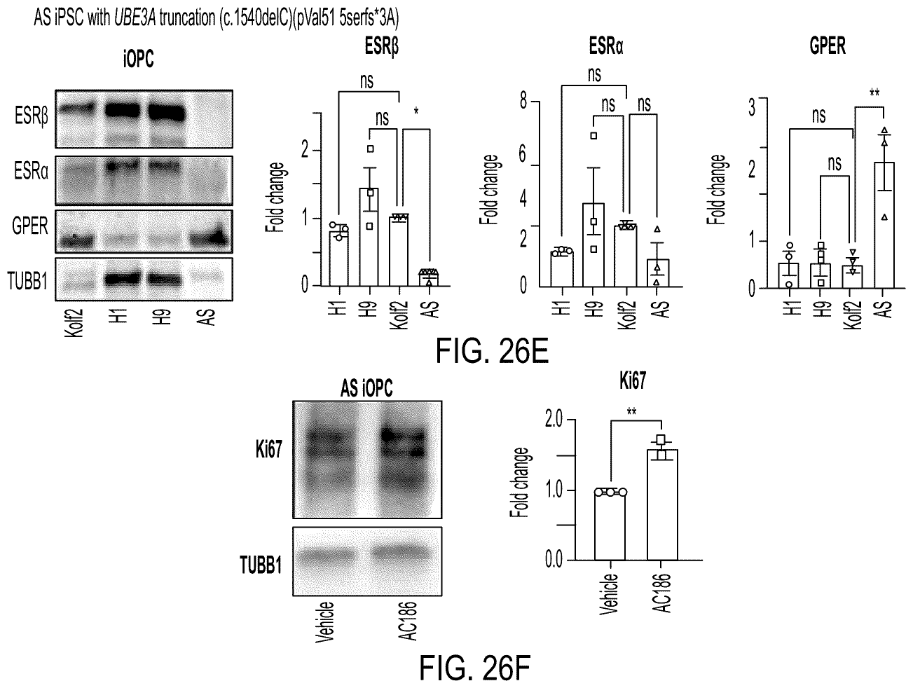

ESR-β functionality was further assessed during oligodendrocyte differentiation by comparing the effects of AC-186 with clemastine—a compound known to promote oligodendrocyte differentiation. While clemastine enhanced differentiation without affecting proliferation in UBE3A-deficient iOLs. AC-186 showed no impact on differentiation (FIG. 25D), suggesting that the impaired ESR-β signaling specifically affects the proliferation stage (iOPC) but not the differentiation into myelinating oligodendrocytes. These findings, aligned with the prior analyses showing a decline in UBE3A levels in mature oligodendrocytes (FIG. 19B-19C), suggest that UBE3A may play a lesser role in the differentiation process for myelination. To confirm this, the timing of UBE3A depletion was adjusted across different stages of oligodendrocyte development, transitioning from iOPCs to pre-iOL (FIG. 25E, 26C) and onto myelin-producing iOL stages. Initiating UBE3A depletion at the iOPC stage significantly impaired differentiation and myelin production. Initiating depletion after the onset of differentiation also had an impact on these processes (FIG. 25E). Additionally, treatment with AC-186 at various stages revealed that its beneficial effects on differentiation were observed when administered during the iOPC stage (FIG. 26C).

Next, iPSCs derived from an AS patient with a truncation mutation in the UBE3A gene were utilized (FIG. 25F). These UBE3A mutant iOPCs displayed normal morphology and expressed markers confirming their identity and functionality (FIG. 25F). They also reproduced characteristics of UBE3A deficiency, such as decreased cell proliferation (FIG. 25F), reduced oligodendrocyte differentiation (FIG. 26D), and lower ESR-β expression (FIG. 26E). They were treated with the ESR-β agonist AC-186, or UBE3A expression in these cells was restored via lentiviral transduction (FIG. 25G), both of which significantly enhanced the proliferation of these mutant iOPCs, thereby confirming the role of UBE3A and its downstream ESR-β signaling in maintaining OPC self-renewal. Further, these UBE3A loss-of-function iOPCs were differentiated into iOLs and the effects of restoring UBE3A expression and treatments with AC-186 or clemastine were examined. While AC-186 showed no impact on oligodendrocyte differentiation, treatment with clemastine and especially UBE3A transduction positively influenced the differentiation process (FIG. 25H, 26F). These findings robustly support the role of UBE3A in oligodendroglial homeostasis and highlight the specificity of the ESR-β signaling pathway, which appears to have a role in OPC proliferation but not in oligodendrocyte differentiation and myelination.

This study shows that selective ESRβ activation treatment corrects proliferative deficits in mouse models of AS and also ameliorates related behavioral abnormalities.

### Materials and Methods

iPSC Culturing and Generation of Induced Human Brain Cells Including Oligodendroglia, Neurons (IN), Astrocytes (iAs) and Microglia (iMG) The generation of iPSC-derived human oligodendrocyte precursor cells (iOPCs) was carried out according to a previously published protocol by Vulakh and Yang et al., 2023 (Characterizing the Neuron-Glial Interactions by the Co-cultures of Human iPSC-Derived Oligodendroglia and Neurons. Methods Mol. Biol. 2683), which itself was a modification of an earlier protocol by Assetta et al., 2020 (Generation of Human Neurons and Oligodendrocytes from Pluripotent Stem Cells for Modeling Neuron-Oligodendrocyte Interactions. J Vis Exp.). The process began with the control iPSCs, which is a well-characterized control reference line acquired from the NIH-funded iPSC Neurodegenerative Disease Initiative (INDI, (Ramos, D. M., Skarnes, W. C., Singleton, A. B., Cookson, M. R., and Ward, M. E. (2021). Tackling neurodegenerative diseases with genomic engineering: A new stem cell initiative from the NIH. Neuron 109, 1080-1083. 10.1016/j.neuron.2021.03.022)), distributed by the Jackson Laboratory. These iPSCs were maintained in mTeSR plus medium (StemCell Technologies, #05825) and expanded in Stem Flex medium (Fisher Scientific. #A3349401) for the induction of neural progenitor cells (INPC). The iNPCs were derived by treating iPSCs with a commercial SMAD inhibitor medium (StemCell Technologies, #08582) for 7 days (Week 1). Subsequently, NPCs were further induced to become OPCs by a 7-day incubation (Week 2) with a chemically-defined OPC medium: DMEM/F12 medium (Thermo Fisher, #11320033) supplemented by 10 ng/ml PDGF-AA (R&D Systems, #221-AA-025), 1 μM SAG (Tocris, #4366), 5 g/mL N-Acetyl-Cysteine (Millipore Sigma, #A9165-5G), 20 ng/mL bFGF (R&D Systems, #233-FB-025/CF), 1% N2 supplement (Thermo Fisher, #17502048), and 1% B27 supplement (Thermo Fisher, #17504044). The resultant iOPCs were developed into pre-mature oligodendrocytes (pre-iOL) in another 7 days (Week 3) by using the OL medium, based on the OPC medium withdrawing growth factors (PDGF-AA and bFGF) and adding 100 ng/ml T3 (MedChem Express, #HY-A0070A), 100 μM Rolipram (MedChem Express, #HY-16900), 100 nM Clemastine (MedChem Express, #HY-B0298A), and 1 μM cAMP (MedChem Express, #HY-B1511). The additional incubation with OL medium for 7-14 days (Week 4-5) yielded mature, myelinating OLs.

The human neurons were induced by NGN2 forced expression in iPSCs as previously reported in Huang et al., 2017 (ApoE2, ApoE3, and ApoE4 Differentially Stimulate APP Transcription and Abeta Secretion. Cell 168, 427-441.e421). In brief, iPSCs were detached with accutase and plated onto Matrigel-coated 6-well plates (4×10⁴ cells/well) on day-2. Lentiviruses expressing Ngn2 and rtTA were prepared as described below, and added to the ES cells in fresh mTeSR1 medium (StemCell Technologies) on day-1. Doxycycline (2 mg/l, to activate Ngn2 expression) was added on day 0 (DO) in DMEM-F12 medium with N₂ supplement without morphogens. Puromycin (1 mg/l) was added on D1 in fresh DMEM-F12/N2+doxycycline medium for selection up to 24 hours. On D2, differentiating neurons were detached with accutase and re-plated on Poly-D-Lysine (50 ng/ml, ThermoFisher Scientific, #A3890401) and laminin (50 μg/mL, Millipore Sigma, #11243217001)-coated 24-well plates (200,000 cells/well), and maintained in NBA/B-27 medium with no doxycycline until sample harvesting. The generation of human astrocytes from iPSCs is concisely described in Connolly et al., 2023 (Modeling Cellular Crosstalk of Neuroinflammation Axis by Tri-cultures of iPSC-Derived Human Microglia, Astrocytes, and Neurons. Methods Mol Biol 2683, 79-87). Human iPSCs were cultured in mTeSR1 with FGF2, differentiated into NPCs through dual SMAD inhibition, and further refined with Rosette Selection Reagent. NPCs were validated via immunocytochemistry, grown on Matrigel in NPC medium with FGF2, and expanded for up to 14 passages, with a preference for lower passages for differentiation. Forebrain NPCs were then differentiated into astrocytes by seeding them at a density of 15,000 cells/cm\hat{ }2 on Matrigel in astrocyte medium. Careful monitoring of initial seeding density and dissociation was crucial for successful differentiation. Cells were maintained in astrocyte medium, fed every other day for 20-30 days, and sub-cultured at 90-95% confluence. After a 30-day differentiation period, astrocytes could be expanded up to 120 days without FBS, noting that increased passaging could affect cell morphology and function.

The generation of human microglial cells from iPSCs, as detailed by Lehoux et al. 2023 (The Generation and Functional Characterization of Human Microglia-Like Cells Derived from iPS and Embryonic Stem Cells. Methods Mol Biol 2683, 69-78), involves a structured protocol using StemCell Technologies kits. This process encompasses three main phases over 38-44 days: differentiation of hematopoietic precursor cells (HPCs) from iPSCs using the STEMdiff Hematopoietic kit, which involves activating specific signaling pathways to produce primitive HPCs; differentiation of these HPCs into microglia by culturing in a cytokine-rich medium to induce microglial marker expression; and maturation of these microglia in a supplemented medium to promote the acquisition of mature microglial characteristics. Throughout these stages, various assays are employed to confirm the identity and maturity of the generated cells, ultimately yielding functionally characterized microglia-like cells.

### Purification and Culturing of Primary Rat Oligodendrocyte Precursor Cells

OPC purification followed our protocol previously described in Mei et al., 2016 (Identification of the Kappa-Opioid Receptor as a Therapeutic Target for Oligodendrocyte Remyelination. J Neurosci 36, 7925-7935), isolating cells from day 7 postnatal rat cortical hemispheres. The process began with mechanical dissection and papain digestion at 37° C. for 75 minutes, with occasional shaking. After trituration, cells were processed through immunopanning with 0.2% BSA, involving 30 minutes of negative and 45 minutes of positive selection. Selection plates were pre-treated with secondary antibodies and incubated with primary antibodies Ran-2, Gal-C (for negative) and O4 (for positive selection). Trypsin dissociated OPCs from the selection dish, and the reaction was stopped with 30% FBS, achieving >95% purity. OPCs were then plated on poly-1-lysine-coated coverslips in a nutrient-rich medium and incubated at 37° C. and 5% CO2 overnight. Post PDGF-AA removal, OPCs received either compound or control treatments for 2 days, with vehicle controls adjusted to match treatment concentrations.

siRNAs and Gene Silencing

The siRNAs targeting UBE3A (Thermo Fisher, #4390815 and #4390816), estrogen receptors (Thermo Scientific #145909 for ESRβ, #145539 for ESRα, and #4390824 for GPER), and negative control (Thermo Scientific, #AM4615) were obtained commercially. The 10-20 nM final concentration was used with Lipofectamine RNAiMAX transfection reagent (Thermo Scientific #13778075) and incubated for at least 48 hours before the treatments of therapeutic compounds or other reagents.

### CRISPR-sgRNAs and Gene Depletion Via Lentiviruses

The sgRNAs targeting UBE3A, and negative control were obtained from Applied Biological Materials Inc. Targeting sgRNA sequences against UBE3A are: sgRNA1, GCTTCAATGTCGTCAGACTG; sgRNA2, CTACTACCACCAGTTAACTG; sgRNA3, GCTTACCTTGAGAACTCGAA. These sgRNAs were integrated into the backbone of the negative control pLenti-U6-sgRNA-SFFV-Cas9-2A-Puro. All constructs were packaged into lentiviruses and concentrated by Lenti-X Concentrator (Takara Bio #631231). In each experiment, 5-10 μl of concentrated virus was administered into ˜70% confluent iOPCs cultures in 6-well plates. One day post-lentiviral transduction, iOPC cultures were treated with approximately 5 μg/ml Puromycin to isolate those iOPCs which are expressing Cas9/sgRNAs. Following a day of selection, the iOPCs were then seeded on gelatin-coated coverslips placed in 24-well plates at a density of 100,000 cells per well. After another day allowed for cell stabilization, treatment with either the vehicle control (DMSO), AC-186, DPN, or LY500307 commenced at a standard concentration of 10 μM. A final 24-hour period after this treatment saw the cells exposed to 5 μM EdU for three hours, preparing them for subsequent immunofluorescence staining.

### Proliferation Assay by Nuclear Incorporation of 5-Ethynyl-2′-Deoxyuridine (EdU)

The EdU labeling kit from ThermoFisher Scientific (#C10637) was used to trace cell proliferation by incubating iOPCs with 2 μM EdU for three hours before fixation. Following EdU incorporation, cells were fixed in 4% paraformaldehyde, blocked with normal serum, and stained with primary antibodies, Rabbit anti-Ki67 and Mouse anti-UBE3A. These steps were conducted according to the manual of the labeling kit and also detailed immunofluorescence staining protocols described below, to ensure specific detection of the proliferation marker Ki67 and the ubiquitin-protein ligase UBE3A in the cells for subsequent imaging analysis.

### Nanofiber Myelination Induction

The in vitro myelination process was adapted from the method established by Lee et al. 2013 (A rapid and reproducible assay for modeling myelination by oligodendrocytes using engineered nanofibers. Nat Protoc 8, 771-782.), utilizing standardized culturing devices and following the manufacturer's guidelines. iOPCs at a density of approximately 250,000 cells were plated on TECL006 Mimetix Aligned scaffold inserts within a 12-well plate (product number TECL006-8x). The cells were cultured in iOPCs medium and the induction medium for oligodendrocyte lineage, which was reduced in growth factors, was renewed every other day. The siRNAs, including a negative control and one targeting UBE3A, were administered on days 1 and 6. On the 10th day after induction, the cells were fixed using 4% paraformaldehyde in preparation for immunofluorescence staining, which involved the application of a rat anti-MBP antibody to visualize myelination.

### Immunoblotting for Assays of Protein Levels

Cells were collected by fast spin down in centrifuge tubes before lysis in RIPA buffer (ThermoFisher, #89901) supplemented with proteinase inhibitor cocktail (Sigma-Aldrich, #5892791001) on ice. Lysates were then spun down at 15000 rpm for 10 mins, the supernatant was collected and mixed with 4× Laemmle sample buffer (Bio-Rad, #1610747) and cooked 10 mins before SDS-PAGE and transferred with Bio-Rad fast transfer system, the membranes were blocked for one hour in 1× Tris Buffered Saline (TBS) with 1% Casein buffer (Bio-Rad, #1610782) and incubated with primary antibodies overnight at 4 degrees. After 3 times washing in TBST washing buffers (ThermoFisher, #28352), the membranes were incubated with HRP conjugated antibodies and developed with Clarity Max™ Western ECL Substrate (Bio-Rad, #1705062) before being filmed in the iBright imaging system.

Real-Time Quantitative PCR (qPCR)

mRNA samples were obtained with Qiagen™ QIAquick PCR Purification Kit. Quantitative real-time PCR was performed using the CFX96 Touch Deep Well Real-Time PCR Detection System. Data were analyzed using the 2^{-ΔΔ}CT method and are presented as fold changes relative to a control sample after normalization against the expression of the housekeeping gene GAPDH. FAM probes for Ube3a, PLP1, MBP, and GAPDH were purchased from (iDT) Integrated DNA Technologies.

### Immunofluorescence Staining

For immunofluorescence with brain tissue sections, mice were sacrificed under deep anesthesia using Xylazine (10 mg/kg body weight) and Ketamine (100 mg/kg body weight) and perfused through the left cardiac ventricle with ice-cold phosphate-buffered saline (PBS) followed by 4% paraformaldehyde (PFA) in PBS. Whole brains were removed and post-fixed in 4% PFA overnight at 4° C. followed by sequential immersion in 15% and 30% sucrose for cryoprotection. The brain was then dissected and 25 μm coronal sections were prepared with a Cryostat Leica. Sections collected on the charged microscope slides (Globe Scientific #1354W) were processed for immunohistochemical staining as follows. In brief, tissue sections were blocked for 1 hour at room temperature (RT) in 5% goat/donkey serum (G/DS) with 0.3% Triton-X100 in 0.1 M phosphate buffer (0.3% PBST). After blocking, tissue sections were incubated in primary antibody diluted in blocking solution overnight at 4° C. The following day, sections were briefly washed 3 times for 5 min each at RT in PBS followed by incubation with secondary antibodies diluted in blocking solution for 2 hours at RT. Sections were then briefly washed 3 times for 5 min each at RT in PBS and mounted using EMS Shield Mount with Dabco (Electron Microscopy Sciences, #17985-200) mounting media with 4′,6-diamidino-2-phenylindole (DAPI) (Vector Laboratories, #H-1200) to stain cell nuclei.

For the purpose of imaging brain sections, we utilized both the BioTek Lionheart FX Automated Microscope and the Zeiss LSM 880 confocal laser-scanning microscope. The subsequent image analysis and quantification were conducted using NIH ImageJ software, employing the particle and intensity analysis macro for detailed examination. Channels were separated, and images from various experimental groups were thresholded uniformly to identify individual cells. In each brain, 20-30 cells were outlined for analysis. To determine the extent of colocalization, we applied the Zeiss ZEN Microscopy Software's colocalization coefficient analysis tool, which allowed for precise measurement of overlapping signals within the selected cells.

For immunofluorescence with fixed cells in vitro, cultured cells were fixed in 4% PFA for 15 min at 37° C. followed by 0.25% TritonX-100 in PBS for 10 min at RT. Cells were blocked with 2% normal goat/donkey serum in PBS for 1 hour at RT. After blocking, cells were incubated with primary antibody in 2% normal goat/donkey serum overnight at 4° C. The following day, coverslips were then washed with PBS 3 times for 5 min each at RT and then incubated with secondary antibody in 2% normal goat/donkey serum for 2 hours at RT. After washing 3 times for 5 min each with PBS, cells were mounted on glass slides using Vectashield mounting media with DAPI followed by obtaining on BioTek Lionheart FX Automated Microscope and/or Zeiss LSM 880 confocal laser-scanning microscope.

To assess the levels of UBE3A protein within oligodendroglial cells, dual immunofluorescent staining was conducted. This involved the use of a UBE3A antibody sourced from Santa Cruz Biotechnology (sc-10294) and an Olig2 antibody provided by Aves Labs (#OLIG2-0020). The corresponding secondary antibodies were tagged with Alexa 647 and Alexa 488 fluorophores. These procedures were applied to coronal brain sections harvested from postnatal days 7-10, 30, and 42 from both control and Angelman Syndrome (AS) mice. We utilized a 20× objective on a GENS microscope to capture confocal images of UBE3A and Olig2 expression. Subsequent analysis was performed using NIH ImageJ software, where fluorescence intensities, denoted in arbitrary units (a.u.), were measured to determine the relative quantities of UBE3A protein. The quantification process involved delineating areas of interest around Olig2-positive cells. Quantitative analysis of ESR2 and MBP proteins was carried out using a similar method.

The List of Primary and Secondary Antibodies Used in this Study

### Primary Antibodies

Rabbit-anti Olig2, Millipore Sigma #AB9610, IHC 1:500
Chicken anti-Olig2, Aves Labs #OLIG2-0020, IHC 1:1000
Mouse-anti-Ube3a, Santa Cruz Biotechnology #sc-166689, IHC 1:100; WB 1:500
Mouse-anti-Ube3a, Millipore Sigma #E8655, IHC 1:500; WB 1:1000
Mouse-anti-04, R&D Systems MAB1326-SP, IHC 1:1000
Rabbit-anti-ESRβ, ThermoFisher Scientific #PA1-310B, WB 1:1000, IHC 1:250
Rabbit-anti-ESRα, ThermoFisher Scientific #BS-2098R, WB 1:1000
Rabbit-anti-GPER, ThermoFisher Scientific #PA5-28647, WB 1:1000
Mouse-anti-KI67, BD Biosciences #556003, IHC 1:100, WB 1:1000
Rabbit-anti-KI67, ThermoFisher Scientific #MA5-14520, IF 1:200
Mouse-anti-PCNA, Proteintech #60097-1-IG, WB 1:2000
Mouse-anti-GAPDH, Proteintech #60004-1-IG, WB 1:4000
Mouse-anti-tubulin B1, Santa Cruz Biotechnology #sc-166729, WB 1:500
Rabbit-anti-NG2, Millipore Sigma #AB5320C3, IF 1:1000, IHC 1:250.
Rat-anti-MBP, Novus Biologicals #NB600-717, IHC 1:100
Mouse-anti-MAG, Santa Cruz Biotechnology #sc-166849, WB 1:200
Rat-anti-PDGFRa, BD Biosciences #558774, WB 1:200
Rabbit-anti-Phospho-PDGF Receptor α (Tyr754) (23B2), Cell Signaling Technology 2992S, 1:200
Mouse-anti-Myelin CNPase, BioLegend #836404, WB 1:500
Mouse-anti-PLP, Santa Cruz Biotechnology #sc-517649, WB 1:200
Chicken-anti-MBP, Aves Labs MBP, WB 1:100
Mouse-anti-A2B5, Millipore Sigma #MAB312R, IF 1:500
Rabbit-anti-PDGFRa, LSBio #LS-C352658-100, IHC 1:100
Chicken-anti-GFAP, Aves Labs GFAP, IHC/IF 1:1000
Mouse-anti-Nestin, StemCell Tech #60091, IF 1:1000

### Secondary Antibodies

Anti-rabbit IgG, HRP linked antibody, Cell Signaling Technology #7074S, WB 1:3000
Anti-mouse IgG, HRP linked antibody, Cell Signaling Technology #7076S, WB 1:3000
Anti-rat IgG, HRP linked antibody, Cell Signaling Technology #7077, WB 1:3000
Anti-chicken IgG, HRP linked antibody, Aves labs #H-1004, WB 1:3000
Goat anti-Chicken IgY (H+L) Secondary Antibody, Alexa Fluor 647, ThermoFisher Scientific #A-21449, IHC 1:500
Goat anti-Mouse IgG (H+L) Cross Adsorbed Secondary Antibody, Alexa Fluor 488, ThermoFisher Scientific #A-11001, IHC 1:500
Goat Anti-Rabbit IgG Polyclonal Antibody (CF™ 555), Minimal Cross-Reactivity, Biotium #20033, IHC 1:500
Goat anti-Rabbit IgG (H+L) Secondary Antibody, Alexa Fluor 568, ThermoFisher Scientific #A-11011, IHC 1:500

### Transgenic Mice of Angelman Syndrome and Animal Care

Ube3a^{tm1Alb/J} mice were purchased from The Jackson Laboratory (stock #016590, Bar Harbor, ME), a breeding colony was established by crossing male C57BL/6J mice (stock #: 000664) with female paternal deletion of Ube3a, and pup genotype was determined as suggested by Jackson Laboratory. Animal handling and experimental use strictly followed NIH guidelines and protocols approved by the local Institutional Animal Care and Use Committee (IACUC) of Brown University. In all experiments, Mice were age and gender-matched. Multiple liters were used for all experiments. Weaned mice, housed in groups of two to four per cage, were maintained on a 12-h light/dark cycle with food and water ad libitum.

### Rodent Learning Behavior Assays

ESRβ agonist AC-186 treatment in vivo. 10 mg/kg AC-186 was daily IP delivered after dissolved at high concentration (30 mg/ml) in DMSO and further diluted in 20% PEG300. Vehicle groups received the same volume and concentration of diluted DMSO in 20% PEG300. Treatment started at P13 and continued until mice were sacrificed at P42.

In assessing motor learning and coordination, our procedures incorporated the accelerating Rotarod test, as described in the previously published study in Lau and Yang, et al., 2023 (AC-186, a selective nonsteroidal estrogen receptor beta agonist, shows gender specific neuroprotection in a Parkinson's disease rat model. ACS Chem Neurosci 4, 1249-1255). This involved conducting two trials per day for three consecutive days, with a 20-minute rest interval between trials. During each trial, mice were placed on a rod that accelerated from 4 to 40 RPM over a five-minute period, and the time until each mouse fell was recorded. The average duration before falling was tracked and graphed across the three-day span.

For evaluating hippocampus-dependent memory, the protocol described in Lau and Yang, et al., 2023 for the fear conditioning test was followed. Mice were placed in an electrified and sound-attenuated chamber (Coulbourn Instruments, Allentown, PA) which was exposed to a scent (0.1% coconut) diluted in ethanol (70%) on day 1. Mice were habituated to the chamber for 3 min before undergoing three conditioning trials: a tone (5 kHz, 70 dB, 30 s) co-terminating with a foot shock (0.7 mA, 1 s). Conditioning trials were separated by a 30-second interval. Following conditioning, mice rested inside the chamber for 1.5 min before returning to their home cage. A camera linked to Noldus Ethovision XT 11 software was used to measure the amount of time mice spent immobile (“freezing”). Context recall was tested by reintroducing the mouse to the same chamber (same scent, no shock) 24 hours after conditioning and measuring freezing. The cued recall was tested by introducing the mouse to a novel context (different scent, different chamber walls, and floor) 48 hours after conditioning and measuring freezing in response to the same auditory stimulus used during conditioning. Scented ethanol was used to clean the chamber between trials.

The Hindlimb Clasping test, which was administered as delineated in a prior study, Guyenet et al., 2010 (A simple composite phenotype scoring system for evaluating 10 mouse models of cerebellar ataxia. J Vis Exp.), is a neurological evaluation that involves the temporary suspension of mice to observe their hindlimb reflexes, a reliable indicator of central nervous system pathology. This method involves a 10-second observation period during which the mice's hindlimb positions are closely monitored and scored. A score of 0 is assigned if the hindlimbs remain splayed outward away from the abdomen consistently; a score of 1 for one hindlimb retracted toward the abdomen for more than half of the time; a score of 2 if both hindlimbs are partially retracted for the majority of the time; and the maximum score of 3 if both hindlimbs are fully retracted and touching the abdomen for over 50% of the observation time. Post-evaluation, mice are promptly returned to their home environment, and scores are averaged over a three-day period to facilitate comparison.

### Statistical Analysis

The determination of sample sizes for our study was not guided by pre-experimental statistical methods due to the absence of effect size estimates prior to the initiation of experiments. The samples sizes were all described in the figure legends. Detailed descriptions of the sample sizes utilized were included in the legends that go with each figure.

The normality of data distributions was assessed using the Shapiro-Wilk test, where a p-value less than 0.05 was indicative of a non-normal distribution. The normality of the data distribution was routinely determined by a Shapiro-Wilk normality test (p<0.05 indicating a non-normal distribution). For data confirmed to be normally distributed, pairwise comparisons were conducted using Student's t-test, while comparisons involving three or more groups were analyzed using One-way or Two-way ANOVA, supplemented by Tukey's post-hoc test, as specified in the figure legends. In case of non-normally distributed data, we resorted to non-parametric tests, including the Mann-Whitney test or the Kruskal-Wallis test, depending on the data structure. A p-value of less than 0.05 was considered statistically significant. For data that is not normally distributed, non-parametric alternatives, such as Mann-Whitney or Kruskal-Wallis tests. p<0.05 was considered to be statistically significant.

All graphical representations, including bar graphs and summary plots, display data as means±standard error of the mean (SEM), derived from a minimum of three independent biological replicates. Significance was reported as * p<0.05. ** p<0.01. *** p<0.001. **** p<0.0001. GraphPad Prism version 9 (GraphPad Software) was utilized for all statistical methods.

### Other Embodiments

Specific compositions and methods for the treatment of Angelman syndrome and/or autism spectrum disorder have been described. The scope of the invention should be defined by the claims. The detailed description in this specification is illustrative and not restrictive or exhaustive. This invention is not limited to the particular methodology, protocols, and reagents described in this specification and can vary in practice. When the specification or claims recite ordered steps or functions, alternative embodiments might perform their functions in a different order or substantially concurrently. Other equivalents and modifications besides those already described are possible without departing from the concepts described in this specification, as persons having ordinary skill in the biomedical art recognize.

All patents and publications cited throughout this specification are incorporated by reference to disclose and describe the materials and methods used with the technologies described in this specification. The patents and publications are provided solely for their disclosure before the filing date of this specification. All statements about the patents and publications' disclosures and publication dates are from the Applicant's information and belief. The Applicant makes no admission about the correctness of the contents or dates of these documents. Should there be a discrepancy between a date provided in this specification and the actual publication date, then the actual publication date shall control. Should there be a discrepancy between the scientific or technical teaching of a previous patent or publication and this specification, then the teaching of this specification and these claims shall control.

The foregoing written specification is considered sufficient to enable one skilled in the biomedical art to practice the present aspects and embodiments. The present aspects and embodiments are not to be limited in scope by examples provided, since the examples are intended as a single illustration of one aspect and other functionally equivalent embodiments are within the scope of the disclosure. Various modifications besides those shown and described herein will become apparent to those skilled in the biomedical art from the foregoing description and fall within the scope of the appended claims. The advantages and objects described herein are not necessarily encompassed by each embodiment. Those skilled in the biomedical art will recognize or be able to ascertain using no more than routine experimentation, many equivalents to the specific embodiments described herein. Such equivalents are intended to be encompassed by these claims.

What is claimed is:

1. A method of treating Angelman syndrome in a subject, the method comprising administering to the subject an effective amount of a non-steroidal selective estrogen receptor beta agonist.

2. A method of treating autism spectrum disorder in a subject, the method comprising administering to the subject an effective amount of a non-steroidal selective estrogen receptor beta agonist.

3. The method of claim 1, wherein:
(i) the non-steroidal selective estrogen receptor beta agonist has an EC50 for estrogen receptor beta of less than 100 nM, less than 50 nM, less than 20 nM, less than 15 nM, less than 10 nM, less than 9 nM, less than 8 nM, or less than 7 nM; and/or
(ii) the EC₅₀ of the non-steroidal selective estrogen receptor beta agonist for estrogen receptor alpha is 100 times greater, 200 times greater, 300 times greater, 400 times greater, 500 times greater, 750 times greater, or 1000 times greater than the EC₅₀ of the non-steroidal selective estrogen receptor beta agonist for estrogen receptor beta.

4. The method of claim 1, wherein the non-steroidal selective estrogen receptor beta agonist comprises a compound of Formula I: 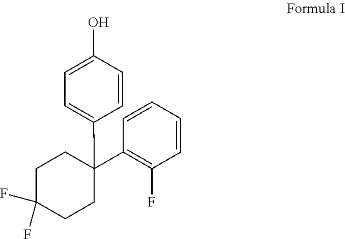 [SMILES: Oc1ccc(C2(c3ccccc3F)CCC(F)(F)CC2)cc1]
or a pharmaceutically acceptable salt thereof.

5. The method of claim 1, wherein the non-steroidal selective estrogen receptor beta agonist comprises a compound of Formula II: 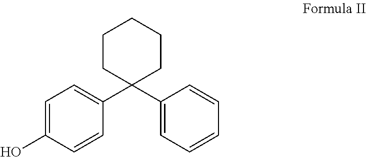 [SMILES: Oc1ccc(C2(c3ccccc3)CCCCC2)cc1]
or a pharmaceutically acceptable salt thereof.

6. The method of claim 1, wherein the non-steroidal selective estrogen receptor beta agonist comprises a compound of Formula III: 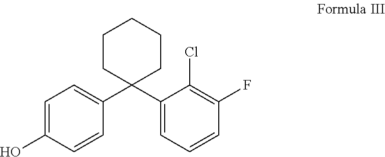 [SMILES: Oc1ccc(C2(c3cccc(F)c3Cl)CCCCC2)cc1]
or a pharmaceutically acceptable salt thereof.

7. The method of claim 1, wherein the non-steroidal selective estrogen receptor beta agonist comprises a compound of Formula IV: 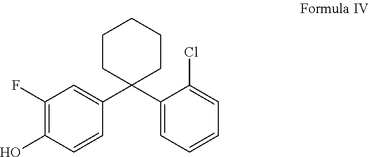 [SMILES: Oc1ccc(C2(c3ccccc3Cl)CCCCC2)cc1F]
or a pharmaceutically acceptable salt thereof.

8. The method of claim 1, wherein the non-steroidal selective estrogen receptor beta agonist is administered in a dose sufficient to reduce oligodendroglial dysfunction in the subject.

9. The method of claim 1, wherein the non-steroidal selective estrogen receptor beta agonist is administered in a dose sufficient to delay onset of neurodevelopmental symptoms in the subject.

10. The method of claim 1, wherein the non-steroidal selective estrogen receptor beta agonist is administered in a dose sufficient to reduce cognitive impairment symptoms in the subject.

11. The method of claim 1, wherein the non-steroidal selective estrogen receptor beta agonist is administered in a dose sufficient to improve motor coordination in the subject.

12. The method of claim 1, wherein the non-steroidal selective estrogen receptor beta agonist is administered in a dose sufficient to reduce motor learning defects in the subject.

13. The method of claim 1, wherein the non-steroidal selective estrogen receptor beta agonist is administered in a dose sufficient to maintain myelination levels or mitigate a decrease in myelination levels in the subject.

14. The method of claim 1, wherein the non-steroidal selective estrogen receptor beta agonist is administered in a dose sufficient to promote proliferation of oligodendrocyte progenitor cells in the subject.

15. The method of claim 1, wherein the non-steroidal selective estrogen receptor beta agonist is administered in a dose sufficient to promote differentiation of oligodendrocyte progenitor cells in the subject.

16. The method of any claim 1 wherein the subject has a deletion in chromosome 15, e.g., in maternal chromosome 15.

17. The method of claim 1, wherein the subject has a mutation in UBE3A.

18. The method of claim 17, wherein the mutation is a truncation mutation.

19. The method of claim 1, wherein:
(i) the subject has an age of 0-1 year, 1-2 years, 2-3 years, 3-4 years, 4-5 years, or greater than 5 years;
(ii) the subject is identified as being at risk of developing Angelman syndrome;
(iii) the subject is identified as being at risk of developing autism spectrum disorder; and/or
(iv) the subject is a human.

20. The method of claim 1, wherein the administration is oral administration, intraperitoneal injection, intravenous administration, or topical administration.
